(19) United States
(12) Patent Application Publication
(10) Pub. No.: US 2016/0326222 A1
(43) Pub. Date: Nov. 10, 2016

(54) CLOSTRIDIUM DIFFICILE VACCINE

(71) Applicant: NATIONAL RESEARCH COUNCIL OF CANADA, Ottawa (CA)

(72) Inventors: Susan M. LOGAN, Ottawa (CA); Susan M. TWINE, Ottawa (CA); Annie AUBRY, Gatineau (CA); Kelly FULTON, Ottawa (CA)

(21) Appl. No.: 15/148,503

(22) Filed: May 6, 2016

Related U.S. Application Data
(60) Provisional application No. 62/158,668, filed on May 8, 2015.

(51) Int. Cl. C07K 14/33; G01N 33/569; A61K 39/08; C07K 16/12

(52) U.S. Cl. CPC C07K 14/33; C07K 16/1282; G01N 33/56911; A61K 39/08; A61K 39/00

(57) ABSTRACT

There are provided compositions and methods for prevention or treatment of Clostridium difficile infection. More specifically, there are provided Clostridium difficile BclA3 spore glycoproteins, as well as glycopeptides and glycans thereof, and their use as a vaccine against Clostridium difficile. Methods of inducing immunity against Clostridium difficile comprising administering a vaccine or an antibody directed against a Clostridium difficile BclA3 spore glycoprotein, or glycopeptide or glycan thereof, are also described.

### RELATED APPLICATIONS

This application claims priority to U.S. Provisional Application No. 62/158,668 filed May 8, 2015, the entire contents of which are hereby incorporated by reference.

### FIELD

The present disclosure provides compositions and methods for prevention or treatment of Clostridium difficile infection. More specifically, the disclosure relates to a Clostridium difficile spore glycoprotein as well as glycopeptides and glycans thereof, and their use as a vaccine against Clostridium difficile.

### BACKGROUND

Clostridium difficile (C. difficile) is a Gram positive, spore forming anaerobe that is a major cause of antibiotic-associated diarrhea. The incidence of C. difficile infection (CDI) has been rapidly increasing in North America and Europe in recent years and this increase in infections has been associated with higher rates of morbidity and mortality. Recent estimates of the incidence of C. difficile associated diarrhea (CDAD) in the U.S. indicate as many as 500,000 cases per year with up to 20,000 deaths (Rupnik, M. et al., Microbiology 7:526-536, 2009; Ananthakrishnan, A. N. Gastroenterology & Hepatology 8:17-26, 2011).

Much C. difficile research has focused on two toxins, TcdA and TcdB, that are produced by C. difficile and that cause tissue damage and a severe inflammatory response, leading in serious cases to potentially lethal pseudomembranous colitis. While toxin activity is recognised as the major virulence factor associated with CDAD, other aspects of C. difficile virulence are less well understood.

Spore production in C. difficile is an integral part of the infectious process. This recalcitrant, dormant form of C. difficile can survive indefinitely outside the host and is known to persist in the hospital environment. It has been demonstrated in mice that antibiotic treatment suppresses the diversity of the gut microbiome and promotes the production of these highly infectious spores, which are then disseminated into the environment (“Supershedder state”) (Lawley, T. D. et al., Infection and immunity 77: 3661-3669, 2009). As such, more recently there has been increased attention on the process of spore formation in C. difficile as well as studies of spore structure and biochemical composition. To date, the focus of studies on spore structure has been to identify spore coat proteins and demonstrate enzymatic activity. Spores are typically pretreated either by enzymatic digestion or sonication to remove the exosporangial layer prior to analysis. However, although considerable progress has been made recently in the analysis of spore coat proteins from C. difficile, the identification and characterisation of both the exosporangial and glycan-containing components is less well advanced, and the surface polysaccharides remain relatively poorly understood.

Current therapies for treatment of CDI target the vegetative phase of the organism's life cycle. Among these treatments are antibiotics such as vancomycin and metronidazole. However, antibiotics are not always effective, and the use of fluoroquinolone antibiotics, such as ciprofloxacin and levofloxacin, has unfortunately led to the emergence of new, highly virulent, and antibiotic resistant strains of C. difficile. Few effective treatments exist for patients with multiple recurrences of C. difficile infection. Fecal bacteriotherapy, or “stool transplant” has been used to re-establish normal intestinal bacterial flora in a patient by transplanting stool from a healthy donor into the patient's intestine (Bakken, J. S., Anaerobe 2009, 15:285-9; Rohlke, F. et al., J. Clin. Gastroenterol. 2010, 44(8):567-70). However, stool transplants can contain pathogenic bacteria and viruses, are not reproducible and controllable, and often carry a psychological stigma for the patient.

Vaccine approaches to date have focused on the A and B toxins and vegetative cell surface proteins (SLPAs) that are produced by metabolically active bacteria. International PCT Application Publication No. WO 2013/084071 describes recombinant fragments of C. difficile TcdA and TcdB and their use in the development of vaccines against C. difficile associated disease, particularly combinations of a ToxB-GT antigen and a TcdA antigen or a ToxA-GT antigen and a TcdB antigen. However, the toxins are produced by vegetative cells rather than spores, and anti-toxin strategies only neutralize the toxin without killing the bacteria.

Carbohydrate-based C. difficile vaccines have been described (Oberli, M. A. et al., Chemistry and Biology 18: 580-588, 2011; Monteiro, M. A. et al., Expert Rev. Vaccines 12: 421-31, 2013; Martin, C. E. et al., J. Am. Chem. Soc. 135: 9713-22, 2013). These studies describe the design and immunogenicity of vaccines composed of raw polysaccharides and conjugates thereof containing the PS-I or PS-II surface glycans from C. difficile. However, such approaches address primary infection by vegetative stage bacteria, but do not target the recalcitrant, dormant, but still infectious, spores.

International PCT Application Publication No. WO 2013/071409 describes a novel lipoteichoic acid (LTA) isolated from C. difficile and its use as a vaccine against CDI and as a diagnostic antigen. Use of LTA-based glycoconjugates as vaccines to combat CDI has also been explored by Cox, A. D. et al. (Glycoconj. 30: 843-55, 2013). However, LTA is found primarily on vegetative cells and cannot be used to target spores specifically.

International PCT Application Publication No. WO 2012/092469 describes compositions and methods for the treatment or prevention of CDI in a vertebrate subject. Compositions containing an antibody or fragment that binds to a C. difficile spore polypeptide or fragment are described, where the spore polypeptide or fragment can be BclA1, BclA2, BclA3, Alr, SIpA paralogue, SIpA HMW, CD1021, lunH, Fe—Mn—SOD, or FliD. Methods of reducing or preventing CDI in a subject are also described, comprising administering to the subject a C. difficile spore polypeptide or fragment or variant, which can be BclA1, BclA2, BclA3, Alr, SlpA paralogue, SlpA HMW, CD1021, lunH, Fe—Mn—SOD, or FliD. However, BclA3 glycosylation is not described, and antisera from mice immunized with BclA3 polypeptide show no reactivity with C. difficile spores, suggesting that BclA3 polypeptide is not an effective immunogen.

There is a need for a safe and effective vaccine composition for preventing or treating CDI based on targeting C. difficile spores.

### SUMMARY

It is an object of the present invention to ameliorate at least some of the deficiencies present in the prior art. Embodiments of the present technology have been developed based on the inventors' appreciation that there is a need for improved compositions and methods for prevention and/or treatment of CDI.

The present disclosure relates to a C. difficile spore glycoprotein and uses thereof. More specifically, there is provided herein a C. difficile BclA3 spore glycoprotein, as well as glycopeptides and glycans thereof, and their use as a vaccine to prevent or treat CDI.

According to a first aspect of the invention, there is provided an isolated C. difficile spore BclA3 glycoprotein or a glycopeptide thereof. The C. difficile BclA3 glycoprotein may comprise the full-length protein or an immunogenic glycopeptide thereof. Functionally equivalent or biologically active homologs, fragments, analogs and/or variants thereof are also encompassed.

In an embodiment, an isolated BclA3 glycoprotein or glycopeptide comprises the amino acid sequence set forth in any one of SEQ ID NOs: 1-32, or a homolog, fragment, analog, or variant thereof. In another embodiment, an isolated BclA3 glycoprotein or glycopeptide comprises an amino acid sequence at least about 80%, at least about 85%, at least about 90%, or at least about 95% identical to the amino acid sequence set forth in any one of SEQ ID NOs: 1-32, or a homolog, fragment, analog, or variant thereof. Isolated BclA3 glycoproteins and glycopeptides are linked to BclA3 glycans, as described herein.

In some embodiments, an isolated BclA3 glycoprotein or glycopeptide comprises one or more chain of three or more N-acetyl hexosamine (HexNAc) moieties O-linked through a threonine residue. For example, a BclA3 glycoprotein or glycopeptide may comprise three or more HexNAc moieties, four or more HexNAc moieties, or five or more HexNAc moieties. In an embodiment, each HexNAc moiety has a molecular weight of about 203 Da. In another embodiment, a BclA3 glycoprotein or glycopeptide further comprises a glycan capping moiety at the end of the HexNAc chain. The glycan capping moiety may have a molecular weight of about 203 Da, about 215 Da, about 220 Da, 372 Da, about 374 Da, about 429 Da, about 486 Da, about 462 Da, about 375 Da, about 424 Da, or about 552 Da. In an embodiment, an isolated BclA3 glycoprotein or glycopeptide comprises one or more GlcNAc residue as a component of the glycan. In an embodiment, an isolated BclA3 glycoprotein or glycopeptide comprises a single O-linked N-acetyl glucosamine (GlcNAc) moiety. In yet another embodiment, a an isolated BclA3 glycoprotein or glycopeptide comprises a single HexNAc moiety.

In yet another embodiment, an isolated BclA3 glycoprotein or glycopeptide comprises a glycopeptide having the nLC-MS/MS spectrum shown in any one of FIGS. 3a, 3b, 8a, and 8b. In some embodiments, an isolated BclA3 glycoprotein or glycopeptide consists of a glycopeptide having the nLC-MS/MS spectrum shown in any one of FIGS. 3a, 3b, 8a, and 8b.

In another aspect, there is provided an isolated C. difficile spore BclA3 glycan. A BclA3 glycan may comprise, for example, one or more chain of three or more N-acetyl hexosamine (HexNAc) moieties, optionally comprising a glycan capping moiety at the end of a HexNAc chain. In an embodiment, a BclA3 glycan comprises a single HexNAc moiety.

In some embodiments, a BclA3 antigen is conjugated to a carrier molecule such as, without limitation, a peptide, a protein, a membrane protein, a carbohydrate moiety, a linker, or a combination thereof, or a liposome containing any of the previous carrier molecules. In some embodiments, a conjugated BclA3 antigen comprises a recombinantly synthesized BclA3 glycan conjugated to a carrier protein. In an embodiment, recombinantly synthesized BclA3 glycan is prepared using SgtA glycosyltransferase, e.g., recombinantly expressed SgtA glycosyltransferase.

In an embodiment, an isolated BclA3 glycoprotein or glycopeptide comprises a BclA3 glycan as described herein linked to a protein or peptide consisting of the amino acid sequence set forth in any one of SEQ ID NOs: 1-32, or a homolog, fragment, analog, or variant thereof. In another embodiment, an isolated BclA3 glycoprotein or glycopeptide comprises a BclA3 glycan as described herein linked to a protein or peptide consisting of an amino acid sequence at least about 80%, at least about 85%, at least about 90%, or at least about 95% identical to the amino acid sequence set forth in any one of SEQ ID NOs: 1-32, or a homolog, fragment, analog, or variant thereof.

In yet another aspect, there are provided compositions comprising the isolated BclA3 glycoprotein or glycopeptide, the isolated BclA3 glycan, or the conjugated BclA3 antigen as described herein, and a pharmaceutically acceptable diluent, carrier, or excipient.

In a further aspect, there are provided vaccines for prevention or treatment of C. difficile infection comprising the isolated BclA3 glycoprotein or glycopeptide, the isolated BclA3 glycan, or the conjugated BclA3 antigen described herein.

In a further aspect, there are provided vaccines for prevention or treatment of C. difficile infection comprising the isolated BclA3 glycoprotein or glycopeptide, the isolated BclA3 glycan, or the conjugated BclA3 antigen described herein, and an adjuvant.

In a still further aspect, there are provided compositions comprising an antibody or fragment thereof that binds to a C. difficile spore glycoprotein or fragment thereof, wherein the glycoprotein or fragment thereof comprises BclA3 glycoprotein or a BclA3 glycopeptide; and a pharmaceutically acceptable diluent, carrier, or excipient. The BclA3 glycoprotein or the glycopeptide may comprise the amino acid sequence set forth in any one of SEQ ID NOs: 1-32, or an amino acid sequence at least about 80%, at least about 85%, at least about 90%, or at least about 95% identical to the amino acid sequence set forth in any one of SEQ ID NOs: 1-32. In an embodiment, the BclA3 glycoprotein or the glycopeptide comprises one or more chain of three or more N-acetyl hexosamine (HexNAc) moieties O-linked through a threonine residue. The BclA3 glycoprotein or the glycopeptide may comprise three, four, or five HexNAc moieties, each HexNAc moiety optionally having a molecular weight of about 203 Da. In an embodiment, the BclA3 glycoprotein or the glycopeptide comprises a single HexNAc moiety. In some embodiments, the BclA3 glycoprotein or the glycopeptide further comprises a glycan capping moiety at the end of the HexNAc chain, the glycan capping moiety optionally having a molecular weight of about 203 Da, about 215 Da, about 220 Da, about 372 Da, about 374 Da, about 429 Da, about 486 Da, about 462 Da, about 375 Da, about 424 Da, or about 552 Da. In an embodiment, the BclA3 glycoprotein or glycopeptide comprises one or more GlcNAc residue as a component of the glycan. In an embodiment, the BclA3 glycopeptide has the nLC-MS/MS spectrum shown in any one of FIGS. 3a, 3b, 8a, and 8b.

In an embodiment, there is provided a composition comprising an antibody or fragment thereof that binds to a C. difficile spore BclA3 glycan and a pharmaceutically acceptable diluent, carrier, or excipient.

In another aspect, there is provided an isolated antibody or fragment thereof specific for C. difficile spores. In an embodiment, the isolated antibody or fragment thereof is specific for a C. difficile spore BclA3 glycoprotein or glycopeptide or glycan thereof. The isolated antibody or fragment may bind specifically to one or more of a BclA3 glycoprotein, a BclA3 glycopeptide, and a BclA3 glycan. In an embodiment, the BclA3 glycoprotein or glycopeptide thereof comprises the amino acid sequence set forth in SEQ ID NOs: 1-32. In another embodiment, the BclA3 glycoprotein or glycopeptide thereof comprises an amino acid sequence at least about 80 to about 95% identical to any one of the amino acid sequences set forth in SEQ ID NOs: 1-32.

In some embodiments, the antibody or fragment thereof is a polyclonal antibody. In alternative embodiments, the antibody or fragment thereof is a monoclonal antibody. The antibody or fragment thereof may be humanized, human, or chimeric. In some embodiments, the antibody or fragment thereof comprises a whole immunoglobulin molecule; a single-chain antibody; a single-chain variable fragment (scFv); a single domain antibody; an Fab fragment; an F(ab′)₂ fragment; or a disulfide-linked Fv (di-scFv). The antibody or fragment thereof may comprise a heavy chain immunoglobulin constant domain selected from human IgM, human IgG1, human IgG2, human IgG3, human IgG4, and human IgA1/2. Further, the antibody or fragment thereof may comprise a light chain immunoglobulin constant domain selected from human Ig kappa and human Ig lambda. In some embodiments, the antibody or fragment binds to an antigen with an affinity constant of at least about 10⁹ M or at least about 10¹⁰ M.

In some embodiments, compositions provided herein further comprise a second agent for preventing or treating C. difficile infection. In some embodiments, the second agent comprises, without limitation, one or more of: an antibody that binds to toxin A; an antibody that binds to toxin B; an antibody that binds to LTA; an antibody that binds to PS-I; an antibody that binds to PS-II; an antibody that binds to a C. difficile vegetative cell surface protein; and, an antibody that binds to a C. difficile spore cell surface protein selected from BclA1, BclA2, Alr, SIpA paralogue, SIpA HMW, CD1021, lunH, Fe—Mn—SOD, and FliD. In another embodiment, the second agent comprises an antibiotic such as, without limitation, metronidazole or vancomycin.

In another aspect, there are provided methods for preventing or treating C. difficile infection comprising administering to a subject the BclA3 antigens, conjugated antigens, compositions, vaccines, or antibodies or fragments thereof described herein, such that C. difficile infection is prevented or treated in the subject. Methods of inducing immunity against C. difficile infection in a subject, such that C. difficile infection is prevented or treated in the subject, are also provided.

A composition, vaccine, antibody or fragment thereof may be administered intravenously, subcutaneously, intramuscularly, or orally. In some embodiments, a composition, vaccine, antibody or fragment thereof is administered in combination with a second agent for preventing or treating C. difficile infection. The second agent may be administered concomitantly with the composition, vaccine, antibody or fragment thereof, or they may be administered sequentially, i.e., one before the other.

Use of a C. difficile spore BclA3 glycoprotein, glycopeptide, glycan or conjugated BclA3 antigen in the manufacture of a vaccine for prevention or treatment of C. difficile infection is also provided.

In another aspect, there is provided an isolated C. difficile SgtA glycosyltransferase and use thereof for preparation of a C. difficile spore antigen, e.g., a C. difficile spore BclA3 glycan. In an embodiment, SgtA glycosyltransferase is recombinantly expressed and used for recombinant BclA3 glycan production.

In yet another aspect, there are provided kits for preventing or treating CDI, comprising one or more C. difficile BclA3 spore antigen, antibody, composition, and/or vaccine as described herein. Instructions for use or for carrying out the methods described herein may also be provided in a kit. A kit may further include additional reagents, solvents, buffers, adjuvants, etc., required for carrying out the methods described herein. Kits for diagnosing CDI in a subject or for determining that a subject is at risk of recurrence of CDI comprising reagents for detecting the presence of one or more of a BclA3 glycoprotein, glycopeptide, and glycan in a subject are also provided.

In an aspect, there are provided diagnostic methods for detecting the presence of C. difficile in a subject. In an embodiment, diagnostic methods for detecting the presence of C. difficile spores in a subject are provided. In some embodiments, there are provided methods of detecting the presence of C. difficile in a subject comprising obtaining a stool sample from the subject and assaying the stool sample for the presence of a BclA3 glycoprotein, glycopeptide, and/or glycan thereof, wherein the presence of the BclA3 glycoprotein, glycopeptide, and/or glycan thereof in the stool sample indicates the presence of C. difficile and/or C. difficile spores in the subject. In some embodiments, the assay comprises an immunoassay. There are further provided uses of the BclA3 antigens and uses of the antibodies or fragments described herein for detecting the presence of C. difficile and/or C. difficile spores in a subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to preferred embodiments of the present invention, and in which:

FIG. 1 is a schematic diagram showing the genetic organization of the putative C. difficile exosporium glycoprotein genes and related glycosyltransferase gene. (a) Bacillus anthracis Sterne strain has been shown to possess two exosporium glycoprotein genes, BAS 1130 and 2281 (coloured black in the figure) denoted bclA and bclB. A glycosyltransferase has also been identified lying adjacent to bclA (BAS 1131, denoted white). Genetic organisation of the bcl homologs (black arrows) and putative glycosyl transferase sgtA (white arrows) in C. difficile 630 (b), R20291 (c) and QCD-32g58 (d) are shown.

FIG. 2 shows NuPAGE gel analysis of C. difficile endospore surface protein extracts. (a) Silver stained 3-8% NuPage, (b) Pro-Emerald Q glycostain 3-8% NuPage. Lane 1: HiMark Prestained molecular weight marker; Lane 2: 630Δerm spore surface extract; Lane 3: R20291 spore surface extract; Lane 4: QCD-32g58 spore surface extract. Arrows indicate regions of the gel that were excised, enzymatically digested, and analysed by nLC-MS/MS.

FIG. 3 shows mass spectrometry analysis of peptides from proteinase K digestion of C. difficile R20291 endospore surface extracts. (a) nLC-MS/MS spectrum of the doubly protonated glycopeptide ion at m/z 811.8 is shown. Peptide type y and b ions were visible and gave the peptide sequence AGLIGPTGATGV, a peptide from the BclA3 protein. The spectrum was dominated in the high m/z region by sequential neutral losses of 203 Da, with the unmodified peptide ion observed at m/z 1013.5. Combined with the observed intense glycan oxonium ion at m/z 204, with neutral losses of water to give glycan related ions at m/z 186 and 168, this spectrum suggested the peptide to be modified with a chain of 3 HexNAc moieties. (b) nLC-MS/Ms spectrum of a doubly protonated glycopeptides ion at m/z 1129 is shown. Peptide type y and b ions corresponded to a sequence of TGPTGATGADGITGP, corresponding to the BclA3 protein. The high m/z region of the spectrum was dominated by sequential neutral losses of 374-203-203-203. An intense oxonium ion was observed at m/z 375 and a very weak oxonium ion at m/z 204 (not indicated). Glycan related fragment ions were observed at m/z 300 and 272. (c) peptide sequence coverage map of BclA3 protein homolog from spore surface protein extraction, band 1 is shown. Boldface and underlining indicates peptides modified with glycan moieties. Two of the peptides shown to be modified with glycan are shown in bold grey text to indicate that the amino acid sequences appear in the BclA3 protein more than once. A dotted underline indicates a glycopeptide sequence that is common to both BclA3 and BclA2 proteins.

FIG. 4 shows immunofluorescence of anti-β-O-GlcNAc binding to spores. (A) Wild type spores of strains of C. difficile, from a range of ribotypes and geographical locations are shown. 630Δerm, GlcNAc binding at poles is marked with arrows. (B) sgtA mutant spores of strains R20291 and 630Δerm are shown. (A) and (B), top row of column, merged image of phase contrast, DAPI and FITC; 2^{nd} row DAPI channel only; 3^{rd} row FITC channel only; 4^{th} row phase contrast only. GlcNAc was visualised with mouse anti-β-O-GlcNAc and anti-mouse IgM-FITC conjugate. (C) shows percentage of spores reacting with anti-β-O-GlcNAc after 7 days of growth. At least 100 spores were counted in triplicate on three independent occasions for anti-6-O-GlcNAc binding to surface, analysed by immunofluorescence microscopy.

FIG. 5 shows restoration of anti-GlcNAc reactivity through complementation. A western blot of 72 hour plate grown cultures run on 3-8% Nu-PAGE gel is shown. Complemented strains were induced with 500 ng anhydrotetracycline. Lane 1: HiMark (Invitrogen); Lane 2: R20291; Lane 3: R20291ΔCDR3194; Lane 4: R20291Δ3194p3350; Lane 5: 630Δerm; Lane 6: 630Δ3350; Lane 7: 630Δ3350p3350.

FIG. 6 shows resistance of R20291 wild type and ΔsgtA spores to 80° C. for 20 minutes. Spores were incubated for 20 minutes in a water bath at 80° C. and then cfu/ml was determined. Percentage survival was calculated by comparing inocula to post heat treatment. Statistical analysis is t-test with Welch's correction, p=<0.0001.

FIG. 7 shows adherence and invasion of J774A.1 macrophage cells. Percentage of spores adhering to or internalised into J774A.1 macrophages after 30 minutes incubation at 37° C. 5% CO₂ is shown. Percentage was calculated based on known MOI and final adhered spores. Fifty J774A.1 cells were counted in triplicate on three independent occasions. Statistical analysis is t-test with Welch's correction (* p=<0.05; *** p=<0.0001).

FIG. 8 shows gel electrophoresis and mass spectrometry analyses of C. difficile QCD-32g58 endospore cell surface protein extract. (a) shows strain QCD-32g58; the MSMS spectrum of the doubly charged precursor ion at m/z 927.4. The y and b ion sequence corresponded to the peptide sequence ³⁰⁷IGPTGATGVTGADGA³²³ from putative exosporium protein (CdifQ_040500019311) with modification with three putative HexNAc residues. (b) shows the MSMS spectrum of the doubly charged peptide precursor on at m.z 1088.5 gave a series of peptide y and b ions, corresponding to the putative exosporial peptide ³⁰⁴AGLIGPTGATGV³¹⁷. Neutral losses corresponding to three HexNac moieties and an unknown glycan of 552 Da were observed in the high m/z region of the spectrum. This gave a total mass excess of 1162 Da. De novo sequencing of the resulting MSMS spectra showed peptides corresponding to a putative exosporium glycoprotein (CdifQ_040500019311). (c) A total of nine glycopeptides were identified, corresponding to 17-21% sequence coverage.

FIG. 9 shows immunofluorescence of anti-β-O-GlcNAc binding to vegetative cells. (a) 630Δerm; (b) R20291, comparing wild type to respective mutant strains (a) ΔCD3350 (b) ΔCDR3194. Left hand column shows FITC labelling only; right hand column shows merged images of FITC, DAPI and transmitted light channels. GlcNAc was visualised with mouse anti-6-O-GlcNAc and anti-mouse IgM-FITC conjugate.

FIG. 10 shows RT-PCR analysis demonstrating co-transcription of CD3350 and CD3349. Upper panel shows expected size of each product with primer pairs P1 (CD3350), P2 (intergenic region) and P3(CD3349); lower panel shows agarose gel analysis of products. RT lanes: RT-PCR was performed using total RNA from C. difficile 630 cells. RNA lanes: standard PCR reaction was performed with same primers using total RNA to demonstrate no contaminating DNA in RNA samples. M: 500 bp DNA marker.

FIG. 11 shows restoration of anti-GlcNAc reactivity through complementation. 72 hour plate grown cultures of (a) 630Δerm, (b) R20291, comparing wild type to ΔCD3350/ΔCDR3194 and ΔCDR3194p3350; complements were induced with 500 ng anyhdrotetracycline. Merged images of FITC, DAPI and transmitted light channels are shown. GlcNAc was visualised with mouse anti-β-O-GlcNAc and anti-mouse IgM-FITC conjugate.

FIG. 12 shows glycostaining of C. difficile spore surface extracts. Surface extracts were run on 3-8% Tris-Acetate NuPAGE prior to glycostaining with Pro-Emerald Q. Lane 1: 630Δerm; Lane 2: 630ΔsgtA; Lane 3: R20291; Lane 4: R20291ΔsgtA; Lane 5: QCD-32g58.

FIG. 13 shows resistance assays with (a) lysozyme and (b) ethanol. (a) R20291 WT and sgtA spores were incubated with 250 μg/ml lysozyme for 1 hour at 37° C. and then percentage survival was calculated. (b) R20291 WT and ΔsgtA spores were incubated in 70% ethanol for 20 minutes at room temperature and then percentage survival was calculated. Assays were performed in triplicate on three independent occasions. Statistical analysis is t-test with Welch's correction (* p=<0.05).

FIG. 14 shows a graph illustrating results from an ELISA assay in which CD5 rabbit polyclonal serum (▴) and preimmune serum (▪) were tested against viable R20291 spores.

FIG. 15 shows a Western blot of spore surface extracts. Lane 1: R20291 spore extract, CD5 preimmune serum; Lane 2: R20291 sgtA spore extract, CD5 preimmune serum; Lane 3: R20291 spore extract, CD5 immune serum; Lane 4: R20291 sgtA spore extract, CD5 immune serum.

### DETAILED DESCRIPTION

The present disclosure relates to the identification and characterization of the BclA3 homolog from C. difficile as a major component of the C. difficile spore surface. We report herein that C. difficile BclA3 is a surface associated glycoprotein modified with a novel oligosaccharide, specifically an O-linked glycan structure. Further, we have demonstrated that antibodies raised against the spore BclA3 glycoprotein recognize C. difficile spores and spore surface extracts. In addition, a glycosyltransferase gene involved in the biosynthesis of surface-associated glycan components was identified, and immunoreactivity of antibodies raised against the spore BclA3 glycoprotein was abrogated in glycosyltransferase mutants. Reactivity of a β-O GlcNAc specific antibody with glycan structures on the C. difficile spore surface confirmed the presence of GlcNAc residue(s) as a component of spore glycan. We have thus demonstrated the immunogenicity and significance of the BclA3 glycan structure and identified the BclA3 glycoprotein as a key immunogen for C. difficile spores.

### BclA3 Spore Antigens

There is provided herein a C. difficile spore antigen comprising a BclA3 glycoprotein, or an immunogenic glycopeptide or glycan thereof. The terms “BclA3 spore antigen”, “spore BclA3 antigen”, “BclA3 antigen”, “C. difficile spore antigen”, and “C. difficile BclA3 spore antigen” are used interchangeably herein to refer to immunogenic molecules comprising a C. difficile BclA3 glycoprotein, a glycopeptide thereof, a glycan thereof, and/or a conjugate thereof, as well as homologs, analogs, variants, and/or fragments thereof. It should be understood that any immunogenic BclA3 glycoprotein, glycopeptide, glycan, conjugate, or homolog, analog, variant, or fragment or portion thereof, is encompassed by the present invention, and may be used in compositions and methods provided herein.

The term “glycoprotein” is used herein to refer to a protein that is post-translationally modified to include glycosylation, i.e., linkage to one or more carbohydrates. The term “glycan” is used to refer to the carbohydrate moiety of a glycoprotein, i.e., the carbohydrates attached to the protein in a glycoprotein. The terms “glycopolypeptide” and “glycopeptide” are used interchangeably herein to refer to a polymer of amino acids attached to one or more carbohydrates post-translationally. As used herein, the term “glycoprotein” generally refers to a full-length protein, e.g., the full-length BclA3 glycoprotein, and the term “glycopeptide” is used to refer to a shorter glycosylated fragment or portion thereof.

As used herein, the term “BclA3 glycoprotein” refers to a full-length glycosylated BclA3 protein from C. difficile such as, without limitation, a glycoprotein having the amino acid sequence set forth in SEQ ID NO: 1 or SEQ ID NO: 24. Many different strains of C. difficile are known and the glycoproteins expressed by different strains may vary slightly in their amino acid sequences and/or their glycan structures. However, the BclA3 glycoprotein provided herein is not meant to be limited to the glycoprotein expressed by any particular strain. It is intended that homologs, variants, fragments, and analogs are encompassed by the present technology. In an embodiment, a BclA3 glycoprotein comprises the BclA3 homolog in a C. difficile strain, such as but not limited to the strains listed in Table 5.

As used herein, the term “BclA3 glycopeptide” refers to an immunogenic and glycosylated peptide fragment or portion of a BclA3 glycoprotein. In some embodiments, a BclA3 glycopeptide has the amino acid sequence set forth in any one of SEQ ID NOs: 2-23 and 25-32. In an embodiment, a BclA3 glycopeptide comprises the amino acid sequence set forth in any one of SEQ ID NOs: 2-23 and 25-32. In another embodiment, a BclA3 glycopeptide consists of the amino acid sequence set forth in any one of SEQ ID NOs: 2-23 and 25-32, the amino acid sequence being O-linked to a glycan, e.g., a chain of three or more N-acetyl hexosamine (HexNAc) moieties.

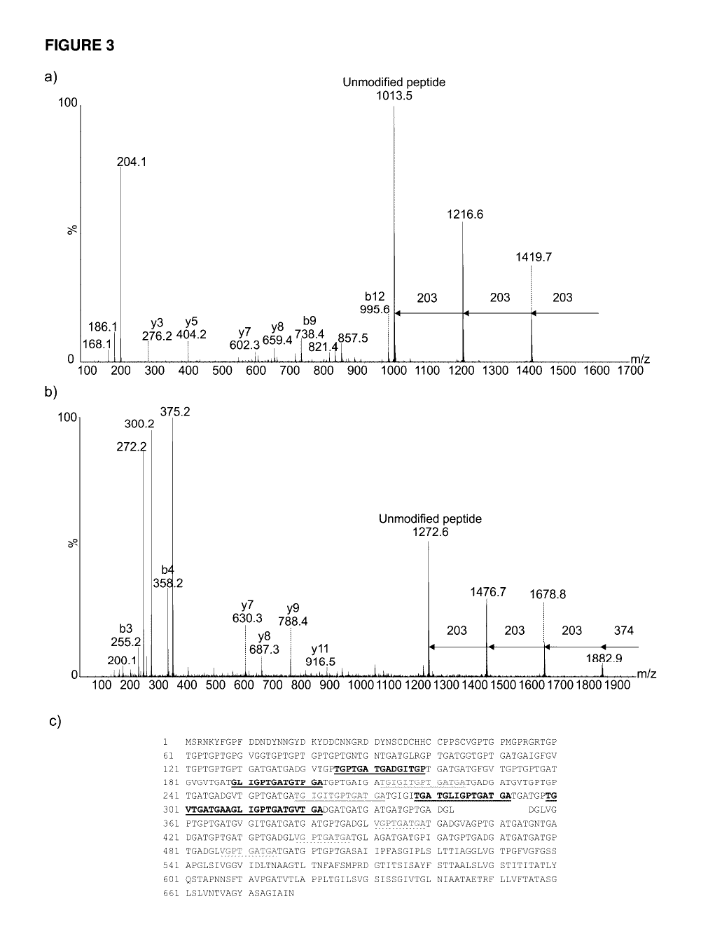

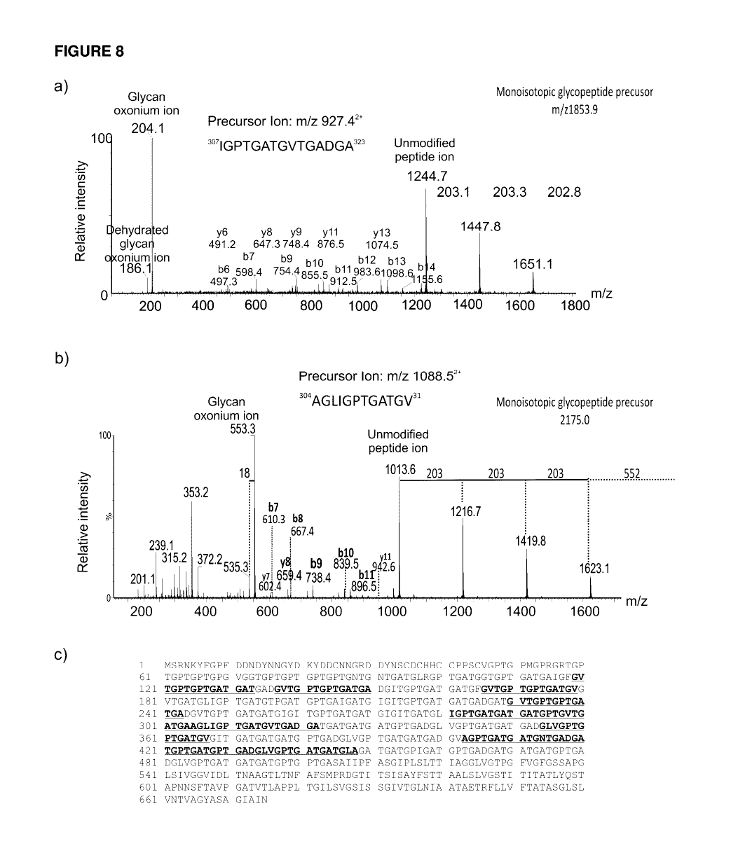

In some embodiments, a BclA3 spore antigen comprises a BclA3 glycoprotein or glycopeptide having the amino acid sequence set forth in any one of SEQ ID Nos: 1-32 and one or more BclA3 glycan, the BclA3 glycan comprising a chain of three or more N-acetyl hexosamine (HexNAc) moieties. In an embodiment, the BclA3 glycan is O-linked, e.g., O-linked through a threonine residue to the BclA3 protein or peptide. In one embodiment, a BclA3 glycan comprises three HexNAc moieties. In another embodiment, a BclA3 glycan comprises five HexNAc moieties. In an embodiment, each HexNAc moiety has a molecular weight of about 203 Da. In some embodiments, a BclA3 glycan further comprises a glycan capping moiety at the end of a HexNAc chain. The glycan capping moiety may have a molecular weight of, for example, about 203 Da, about 215 Da, about 220 Da, about 372 Da, about 374 Da, about 429 Da, about 486 Da, about 462 Da, about 375 Da, about 424 Da, or about 552 Da. In an embodiment, a BclA3 glycan comprises one or more GlcNAc residue. In a particular embodiment, a BclA3 glycopeptide comprises or consists of a glycopeptide having the nLC-MS/MS spectrum shown in any one of FIGS. 3a, 3b, 8a, and 8b.

In some embodiments, a BclA3 spore antigen comprises at least one carbohydrate chain comprising an O-linked N-acetyl glucosamine (GlcNAc) moiety. In an embodiment, a BclA3 spore antigen comprises a single O-linked N-acetyl glucosamine (GlcNAc) moiety.

The amino acid sequences of the protein/peptide portion of exemplary BclA3 glycoproteins and glycopeptides are given in Table 1.

TABLE 1
Amino acid sequences of exemplary BclA3 glycoproteins and glycopeptides.

| SEQ | | |
|---|---|---|
| ID | | |
| NO. | Amino acid sequence | Source |
| 1 | MSRNKYFGPFDDNDYNNGYDKYDDCNNGRDDYNSCDCHHCCPPSCVGPTGPMGPRGRTGPTGPT | BclA3 |
| | GPTGPGVGGTGPTGPTGPTGPTGNTGNTGATGLRGPTGATGGTGPTGATGAIGFGVTGPTGPTG | glycoprotein |
| | PTGATGATGADGVTGPTGPTGATGADGITGPTGATGATGFGVTGPTGPTGATGVGVTGATGLIG | (full-length), |
| | PTGATGTPGATGPTGAIGATGIGITGPTGATGATGADGATGVTGPTGPTGATGADGVTGPTGAT | C. difficile |
| | GATGIGITGPTGATGATGIGITGATGLIGPTGATGATGATGPTGVTGATGAAGLIGPTGATGVT | R20291 strain |
| | GADGATGATGATGATGPTGADGLVGPTGATGATGADGLVGPTGPTGATGVGITGATGATGATGP | |
| | TGADGLVGPTGATGATGADGVAGPTGATGATGNTGADGATGPTGATGPTGADGLVGPTGATGAT | |
| | GLAGATGATGPIGATGPTGADGATGATGATGPTGADGLVGPTGATGATGATGPTGPTGASAIIP | |
| | FASGIPLSLTTIAGGLVGTPGFVGFGSSAPGLSIVGGVIDLTNAAGTLTNFAFSMPRDGTITSI | |
| | SAYFSTTAALSLVGSTITITATLYQSTAPNNSFTAVPGATVTLAPPLTGILSVGSISSGIVTGL | |
| | NIAATAETRFLLVFTATASGLSLVNTVAGYASAGIAIN | |
| 2 | AGLIGPTGATGV | C. difficile |
| | | R20291 and |
| | | QCD-32g58 |
| | | strains |
| 3 | TGPTGATGADGITGP | C. difficile |
| | | R20291 strain |
| 4 | VGPTGATGA | C. difficile |
| | | R20291 strain |
| 5 | GLIGPTGATGTPGA | C. difficile |
| | | R20291 strain |
| 6 | TGATGLIGPTGATGA | C. difficile |
| | | R20291 strain |
| 7 | TGIGITGPTGATGA | C. difficile |
| | | R20291 strain |
| 8 | TGIGITGPTGA | C. difficile |
| | | R20291 strain |
| 9 | GLIGPTGATGVTGA | C. difficile |
| | | R20291 strain |
| 10 | TGVTGATGAAGLIGP | C. difficile |
| | | R20291 strain |
| 11 | TGATGLIGPTGATGA | C. difficile |
| | | R20291 strain |
| 12 | IGPTGATGTPGATGPTGA | C. difficile |
| | | R20291 strain |
| 13 | TGPTGATGPTGADGL | C. difficile |
| | | R20291 and |
| | | QCD-32g58 |
| | | strains |
| 14 | GVTGPTGPTGPTGATGA | C. difficile |
| | | R20291 strain |
| 15 | GVTGPTGPTGATGV | C. difficile |
| | | R20291 strain |
| 16 | VGPTGATGATGADGL | C. difficile |
| | | R20291 strain |
| 17 | VGPTGPTGATGV | C. difficile |
| | | R20291 strain |
| 18 | IGPTGATGTPGATGPTGA | C. difficile |
| | | R20291 strain |
| 19 | IGPTGATGVTGADGA | C. difficile |
| | | R20291 strain |
| 20 | VGPTGATGATGL | C. difficile |
| | | R20291 and |
| | | QCD-32g58 |
| | | strains |
| 21 | VGPTGATGATGADGV | C. difficile |
| | | R20291 strain |
| 22 | VGPTGPTGATGV | C. difficile |
| | | R20291 strain |
| 23 | ATASGLSLVNTVA | C. difficile |
| | | R20291 strain |
| 24 | MSRNKYFGPFDDNDYNNGYDKYDDCNNGRDDYNSCDCHHCCPPSCVGPTGPMGPRGRTGPTGPT | BclA3 |
| | GPTGPGVGGTGPTGPTGPTGPTGNTGNTGATGLRGPTGATGGTGPTGATGAIGFGVTGPTGPTG | glycoprotein |
| | ATGATGADGVTGPTGPTGATGADGITGPTGATGATGFGVTGPTGPTGATGVGVTGATGLIGPTG | (full-length), |
| | ATGTPGATGPTGAIGATGIGITGPTGATGATGADGATGVTGPTGPTGATGADGVTGPTGATGAT | C. difficile |
| | GIGITGPTGATGATGIGITGATGLIGPTGATGATGATGPTGVTGATGAAGLIGPTGATGVTGAD | QCD-32g58 |
| | GATGATGATGATGPTGADGLVGPTGATGATGADGLVGPTGPTGATGVGITGATGATGATGPTGA | strain |
| | DGLVGPTGATGATGADGVAGPTGATGATGNTGADGATGPTGATGPTGADGLVGPTGATGATGLA | |
| | GATGATGPIGATGPTGADGATGATGATGPTGADGLVGPTGATGATGATGPTGPTGASAIIPFAS | |
| | GIPLSLTTIAGGLVGTPGFVGFGSSAPGLSIVGGVIDLTNAAGTLTNFAFSMPRDGTITSISAY | |
| | FSTTAALSLVGSTITITATLYQSTAPNNSFTAVPGATVTLAPPLTGILSVGSISSGIVTGLNIA | |
| | ATAETRFLLVFTATASGLSLVNTVAGYASAGIAIN | |
| 25 | TGPTGVTGATGA | C. difficile |
| | | QCD-32g58 |
| | | strain |
| 26 | GVTGPTGPTGATGA | C. difficile |
| | | QCD-32g58 |
| | | strain |
| 27 | GVTGPTGPTGATGV | C. difficile |
| | | QCD-32g58 |
| | | strain |
| 28 | TGPTGADGL | C. difficile |
| | | QCD-32g58 |
| | | strain |
| 29 | GLVGPTGPTGATGV | C. difficile |
| | | QCD-32g58 |
| | | strain |
| 30 | AGPTGATGATGNTGADGA | C. difficile |
| | | QCD-32g58 |
| | | strain |
| 31 | TGPTGATGPTGADGLVGPTGATGATGLA | C. difficile |
| | | QCD-32g58 |
| | | strain |
| 32 | IGPTGATGVTGADGA | C. difficile |
| | | QCD-32g58 |
| | | strain |

Variants, analogs, and fragments of BclA3 glycoproteins and glycopeptides are also encompassed. As used herein, a “variant” refers to an amino acid sequence of the naturally occurring protein or peptide in which a small number of amino acids have been substituted, inserted, or deleted, and which retains the relevant biological activity or function of the starting protein. For example, in the case of an antigen for use in a vaccine, a variant may retain the immunogenic characteristics of the starting protein, sufficient for its intended use in inducing immunity. In the case of an antibody, a variant may retain the antigen-binding properties of the starting protein, sufficient for its intended use in binding specifically to antigen.

In some embodiments, a variant includes one or more conservative amino acid substitutions, one or more non-conservative amino acid substitutions, one or more deletions, and/or one or more insertions. A conservative substitution is one in which an amino acid residue is substituted by another amino acid residue having similar characteristics (e.g., charge or hydrophobicity). In general, a conservative amino acid substitution will not substantially change the functional properties of a protein. Examples of groups of amino acids that have side chains with similar chemical properties include: 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Other conservative amino acid substitutions are known in the art and are included herein. Non-conservative substitutions, such as replacing a basic amino acid with a hydrophobic one, are also well-known in the art.

As used herein, an “analog” refers to an amino acid sequence of the naturally occurring protein in which one or more amino acids have been replaced by amino acid analogs. Non-limiting examples of amino acid analogs include non-naturally occurring amino acids, synthetic amino acids, amino acids which only occur naturally in an unrelated biological system, modified amino acids from mammalian systems, polypeptides with substituted linkages, as well as other modifications known in the art, both naturally occurring and non-naturally occurring. In some embodiments, analogs include modifications which increase glycoprotein or glycopeptide stability. In one embodiment, an analog includes a beta amino acid, a gamma amino acid, or a D-amino acid.

A “fragment” refers to a portion of the starting molecule which retains the relevant biological activity or function (e.g, antigenicity, antigen-binding, immunogenicity) of the starting molecule.

A “biologically active” or “functionally equivalent” fragment, variant, or analog generally retains biological activity or function of the starting molecule, sufficient for use in the present compositions and methods. Thus, a “biologically active” or “functionally equivalent” fragment, variant, or analog may retain the binding specificity, the antigenicity, or the immunogenicity of the starting molecule. In some embodiments, a fragment, variant or analog has at least about 70%, at least about 80%, at least about 85%, at least about 90%, at least about 95%, or at least about 98% sequence identity to the starting molecule (e.g., protein). When referring to an antibody, “functionally equivalent” generally refers to a fragment, derivative, variant, analog, or fusion protein of the antibody that maintains sufficient antigen-binding affinity, specificity and/or selectivity for use in the present compositions and methods. The antigen-binding properties of a functionally equivalent antibody or fragment need not be identical to those of the reference antibody so long as they are sufficient for use in the present compositions and methods for preventing or treating CDI.

Variants, fragments, or analogs may also be modified at the N- and/or C-terminal ends to allow the polypeptide or fragment to be conformationally constrained and/or to allow coupling to an immunogenic carrier.

There are further provided conjugated BclA3 spore antigens comprising a BclA3 antigen conjugated to a carrier molecule. A carrier molecule may be any suitable molecule such as, without limitation, a peptide, a protein, a membrane protein, a carbohydrate moiety, or one or more liposomes loaded with any of the previously recited types of carrier molecules or loaded with a BclA3 antigen itself. Many such carrier molecules are known in the art and may be used in the conjugated BclA3 antigens provided herein. In one embodiment, a conjugated BclA3 antigen comprises a BclA3 antigen conjugated to a carrier protein. In an embodiment, a conjugated BclA3 antigen comprises a BclA3 glycan. In another embodiment, a BclA3 antigen is conjugated to a linker molecule or a protein carrier.

Further, a carrier molecule may be linked to a BclA3 antigen, e.g., a BclA3 glycan, using any suitable method known in the art. For example, a carrier molecule may be linked to a BclA3 antigen by a covalent bond or an ionic interaction, either directly or using a linker. Linkage may be achieved by chemical cross-linking, e.g., a thiol linkage. A carrier protein or peptide may be linked to a BclA3 glycan through, for example, O-linkage of the glycan to a threonine residue in the peptide. Methods for linking glycans to carrier molecules are well-known in the art, as are methods for preparing glycoconjugate vaccines. In an embodiment, a conjugated glycan antigen is prepared by conjugating a recombinantly-synthesized glycan to a carrier protein.

In another embodiment, a spore antigen is produced as a fusion protein or a conjugate that contains other distinct amino acid sequences that are not part of the C. difficile spore antigen sequences disclosed herein, such as amino acid linkers or signal sequences or immunogenic carriers, as well as ligands useful in protein purification, such as glutathione-S-transferase, histidine tag, and staphylococcal protein A. A heterologous polypeptide can be fused, for example, to the N-terminus or C-terminus of a BclA3 peptide or protein. Further, more than one BclA3 peptide can be present in a fusion protein

As used herein, the term “isolated” refers to a molecule that by virtue of its origin or source of derivation (1) is not associated with naturally associated components that accompany it in its native state, (2) is free of other macromolecules (e.g., proteins, glycans) from the same species, (3) is expressed by a cell from a different species, or (4) does not occur in nature. Thus, a glycoprotein, glycopeptide or glycan that is chemically synthesized or synthesized in a cellular system different from the cell from which it naturally originates will be “isolated” from its naturally associated components. A glycoprotein, glycopeptide or glycan may also be rendered substantially free of naturally associated components by isolation, using purification or separation techniques well-known in the art. BclA3 spore antigens used in compositions and methods described herein are generally provided in purified or substantially purified form, i.e., substantially free from other glycopeptides and polypeptides, particularly from other C. difficile or host cell glycopeptides or polypeptides. In some embodiments, BclA3 spore antigens are at least about 50% pure, at least about 60% pure, at least about 70% pure, at least about 80%, at least about 90% pure, or at least about 95% pure (by weight).

BclA3 glycoproteins, glycopeptides and glycans thereof can be prepared by various means (e.g., recombinant expression, purification from cell culture, chemical synthesis, etc.). In some embodiments, a BclA3 spore antigen is chemically synthesized. For example, a glycan may be chemically synthesized and then coupled to a protein or peptide, which protein or peptide may also be chemically synthesized (e.g., using solid phase peptide synthesis) or purified after recombinant expression in a cell line. Alternatively, a BclA3 spore antigen may be purified after recombinant expression in a cell line. For example, a polynucleotide encoding a BclA3 protein or peptide can be introduced into an expression vector that can be expressed in a suitable expression system using techniques well-known in the art, followed by isolation or purification of the expressed protein or peptide. A variety of bacterial, yeast, plant, mammalian, and insect expression systems are available in the art and any such suitable expression system can be used. A glycoprotein or glycopeptide can be expressed in systems, e.g. cultured cells, cell lines, etc., which produce substantially the same postranslational modifications present as when the protein or peptide is expressed natively. Alternatively, a polynucleotide encoding a BclA3 protein or peptide can be translated in a cell-free translation system.

In cases where the expression system or cell line is competent to glycosylate the expressed BclA3 protein or peptide appropriately, then the glycosylated BclA3 spore antigen may be purified from the expression system or cell line. Alternatively, an unglycosylated BclA3 protein or peptide may be linked to a glycan subsequently, after expressing and purifying the protein or peptide from a host cell. For example, a glycan can be chemically synthesized and then coupled to a protein or peptide in vitro. In an embodiment, an unglycosylated BclA3 protein or peptide is incubated with a glycosylation enzyme, e.g., SgtA glycosyltransferase, in appropriate conditions to allow glycosylation of the BclA3 protein or peptide. In an embodiment, recombinantly expressed SgtA glycosyltransferase is used for production of a BclA3 spore antigen, or for production of a recombinant glycan. BclA3 antigen conjugates, e.g, BclA3 glycan conjugates, can be prepared using similar techniques, including without limitation chemical synthesis, recombinant expression, use of recombinantly expressed SgtA glycosyltransferase, and/or a combination thereof, as well as other techniques known in the art.

A spore antigen can also be produced as a fusion protein or a conjugate that contains other distinct amino acid sequences that are not part of the C. difficile spore antigen sequences disclosed herein, such as amino acid linkers or signal sequences or immunogenic carriers, as well as ligands useful in protein purification, such as glutathione-S-transferase, histidine tag, and staphylococcal protein A. A heterologous polypeptide can be fused, for example, to the N-terminus or C-terminus of a BclA3 peptide or protein. Further, more than one BclA3 peptide can be present in a fusion protein.

Many variations of techniques described herein are known in the art and may be used to prepare BclA3 spore antigens.

### Pharmaceutical Compositions and Methods

There are provided herein compositions and methods for the prevention or treatment of C. difficile infection (CDI) in a subject comprising immunogenic BclA3 spore antigens. Compositions and methods for inducing an immune response to C. difficile are also provided. Methods provided herein comprise administration of a C. difficile BclA3 spore antigen (e.g., BclA3 glycoprotein, glycopeptide or glycan thereof), to a subject in an amount effective to induce an immune response against C. difficile spores, thereby reducing, eliminating, preventing, or treating CDI. Compositions and methods are also provided for the generation of antibodies for use in passive immunization against CDI.

C. difficile infection (CDI) is a bacterial infectious disease of the gastrointestinal tract caused by Clostridium difficile (C. difficile), a toxin-producing Gram-positive anaerobic, spore-forming bacillus. As used herein, CDI includes recurrent CDI, which is defined as complete resolution of CDI while on appropriate therapy, followed by recurrence of CDI after treatment has been stopped. CDI is often associated with disorders of the gastrointestinal tract such as dysbiosis, Crohn's disease, ulcerative colitis, enteritis, irritable bowel syndrome, inflammatory bowel disease, diarrhea, antibiotic-associated diarrhea, and diverticular disease. In some embodiments, there are provided compositions and methods for prevention or treatment of disorders of the gastrointestinal tract associated with CDI such as, without limitation, dysbiosis, Crohn's disease, ulcerative colitis, enteritis, irritable bowel syndrome, inflammatory bowel disease, diarrhea, antibiotic-associated diarrhea, and diverticular disease.

The terms “subject” and “patient” are used interchangeably herein to refer to a subject in need of prevention or treatment for CDI or for a disorder of the gastrointestinal tract associated with CDI, including those at risk of contracting CDI for the first time and those at risk of recurrence of CDI. A subject may be a vertebrate, such as a mammal, e.g., a human, a non-human primate, a rabbit, a rat, a mouse, a cow, a horse, a goat, or another animal. Animals include all vertebrates, e.g., mammals and non-mammals, such as mice, sheep, dogs, cows, avian species, ducks, geese, pigs, chickens, amphibians, and reptiles. In an embodiment, a subject is a human.

“Treating” or “treatment” refers to either (i) the prevention of infection or reinfection, e.g., prophylaxis, or (ii) the reduction or elimination of symptoms of the disease of interest, e.g., therapy. “Treating” or “treatment” can refer to the administration of a composition comprising a BclA3 glycoprotein, glycopeptide, or glycan of interest, e.g., C. difficile BclA3 spore antigens, or to the administration of antibodies raised against these antigens. Treating a subject with the composition can prevent or reduce the risk of infection and/or recurrence and/or induce an immune response to C. difficile spores. In some embodiments, spore germination is inhibited or delayed; pathogen burden is reduced; spore colonization is inhibited; and/or spore adherence to the GI tract is blocked in a subject.

Treatment can be prophylactic (e.g., to prevent or delay the onset of the disease, to prevent the manifestation of clinical or subclinical symptoms thereof, or to prevent recurrence of the disease) or therapeutic (e.g., suppression or alleviation of symptoms after the manifestation of the disease). “Preventing” or “prevention” refers to prophylactic administration or vaccination with BclA3 spore antigens or antigen compositions in a subject who has not been infected or who is symptom-free after CDI and at risk of recurrence of CDI.

As used herein, the term “immune response” refers to the response of immune system cells to external or internal stimuli (e.g., antigens, cell surface receptors, cytokines, chemokines, and other cells) producing biochemical changes in the immune cells that result in immune cell migration, killing of target cells, phagocytosis, production of antibodies, production of soluble effectors of the immune response, and the like. An “immunogenic” molecule is one that is capable of producing an immune response in a subject after administration.

“Active immunization” refers to the process of administering an antigen (e.g., an immunogenic molecule) to a subject in order to induce an immune response. In contrast, “passive immunization” refers to the administration of active humoral immunity, usually in the form of pre-made antibodies, to a subject. Passive immunization is a form of short-term immunization that can be achieved by the administration of an antibody or an antigen-binding fragment thereof. Antibodies can be administered in several possible forms, for example as human or animal blood plasma or serum, as pooled animal or human immunoglobulin, as high-titer animal or human antibodies from immunized subjects or from donors recovering from a disease, as polyclonal antibodies, or as monoclonal antibodies. Typically, immunity derived from passive immunization provides immediate protection or treatment but may last for only a short period of time.

In some embodiments, there are provided compositions and methods for active immunization against C. difficile. Compositions and methods are provided for inducing an immune response to C. difficile bacteria in a subject, comprising administering to the subject a C. difficile BclA3 spore antigen and an adjuvant in an amount effective to induce an immune response in the subject. In one embodiment, there is provided a composition comprising an effective immunizing amount of an isolated C. difficile BclA3 spore antigen and an adjuvant, wherein the composition is effective to prevent or treat CDI in a subject in need thereof. In an embodiment, the BclA3 spore antigen comprises one or more BclA3 glycoprotein, glycopeptide, glycan, or conjugated BclA3 antigen (e.g., conjugated BclA3 glycan antigen), as described herein. In an embodiment, an adjuvant is not required, i.e., compositions and methods are provided for inducing an immune response to C. difficile bacteria in a subject, comprising administering to the subject a C. difficile BclA3 spore antigen and a pharmaceutically acceptable carrier, excipient, or diluent, in an amount effective to induce an immune response in the subject.

Adjuvants generally increase the specificity and/or the level of immune response. An adjuvant may thus reduce the quantity of antigen necessary to induce an immune response, and/or the frequency of injection necessary in order to generate a sufficient immune response to benefit the subject. Any compound or compounds that act to increase an immune response to an antigen and are suitable for use in a subject (e.g., pharmaceutically-acceptable) may be used as an adjuvant in compositions, vaccines, and methods of the invention. In some embodiments, the adjuvant may be the carrier molecule (for example, but not limited to, cholera toxin B subunit, liposome, etc.) in a conjugated or recombinant antigen. In alternative embodiments, the adjuvant may be an unrelated molecule known to increase the response of the immune system (for example, but not limited to attenuated bacterial or viral vectors, AMVAD, etc.). In one embodiment, the adjuvant may be one that generates a strong mucosal immune response such as an attenuated virus or bacteria, or aluminum salts. Other suitable adjuvants are well-known to those of skill in the art.

Compositions, formulations and vaccines including one or more BclA3 spore antigen described herein can be prepared by uniformly and intimately bringing into association the antigen and the adjuvant using techniques well-known to those skilled in the art including, but not limited to, mixing, sonication and microfluidation. An adjuvant will typically comprise about 10 to 50% (v/v), about 20 to 40% (v/v), or about 20 to 30% or 35% (v/v) of the composition.

In other embodiments, there are provided compositions and methods for passive immunization comprising an antibody or an antigen-binding fragment thereof specific for a C. difficile spore BclA3 antigen. As used herein, the term “antibody” refers to any immunoglobulin or intact molecule as well as to fragments thereof that bind to a specific antigen or epitope. Such antibodies include, but are not limited to polyclonal, monoclonal, chimeric, humanized, single chain, Fab, Fab′, F(ab′)₂, F(ab)′ fragments, and/or F(v) portions of the whole antibody and variants thereof. All isotypes are emcompassed by this term, including IgA, IgD, IgE, IgG, and IgM.

As used herein, the term “antibody fragment” refers to a functionally equivalent fragment or portion of antibody, i.e., to an incomplete or isolated portion of the full sequence of an antibody which retains the antigen binding capacity (e.g., specificity, affinity, and/or selectivity) of the parent antibody. Non-limiting examples of antigen-binding portions include: (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment, which consists of a VH domain; (vi) an isolated complementarity determining region (CDR); and (vii) a single chain Fv (scFv), which consists of the two domains of the Fv fragment, V_{L} and V_{H}. Other non-limiting examples of antibody fragments are Fab′ fragments; diabodies; linear antibodies; single-chain antibody molecules; and multispecific antibodies formed from antibody fragments. An intact “antibody” comprises at least two heavy (H) chains and two light (L) chains inter-connected by disulfide bonds. Each heavy chain is comprised of a heavy chain variable region (V_{H}) and a heavy chain constant region. The heavy chain constant region is comprised of three domains, CH₁, CH₂ and CH₃. Each light chain is comprised of a light chain variable region (V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the term “monoclonal antibody” or “mAb” refers to a preparation of antibody molecules of single molecular composition. A monoclonal antibody composition displays a single binding specificity and affinity for a particular epitope. A “human monoclonal antibody” refers to antibodies displaying a single binding specificity which have variable and constant regions (if present) derived from human germline immunoglobulin sequences. In one aspect, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell. A “humanized antibody” refers to at least one antibody molecule in which the amino acid sequence in the non-antigen binding regions and/or the antigen-binding regions has been altered so that the antibody more closely resembles a human antibody, and still retains its original binding properties. Humanized antibodies are typically antibody molecules from non-human species having one or more CDRs from the non-human species and a framework region from a human immunoglobulin molecule. The term “chimeric antibody” refers to an antibody in which different portions are derived from different animal species, e.g., an antibody having a variable region derived from a murine mAb and a human immunoglobulin constant region.

As used herein, the term “antigen” refers to a substance that prompts the generation of antibodies and can cause an immune response. The terms “antigen” and “immunogen” are used interchangeably herein, although, in the strict sense, immunogens are substances that elicit a response from the immune system, whereas antigens are defined as substances that bind to specific antibodies. An antigen or fragment thereof can be a molecule (i.e., an epitope) that makes contact with a particular antibody. When a glycoprotein or a fragment thereof is used to immunize a host animal, numerous regions of the glycoprotein can induce the production of antibodies (i.e., elicit the immune response), which bind specifically to the antigen (given regions or three-dimensional structures on the glycoprotein).

The terms “specific for” or “specifically binding” are used interchangeably to refer to the interaction between an antibody and its corresponding antigen. The interaction is dependent upon the presence of a particular structure of the protein recognized by the binding molecule (i.e., the antigen or epitope). In order for binding to be specific, it should involve antibody binding of the epitope(s) of interest and not background antigens, i.e., no more than a small amount of cross reactivity with other antigens (such as other proteins or glycan structures, host cell proteins, etc.). Antibodies, or antigen-binding fragments, variants or derivatives thereof of the present disclosure can also be described or specified in terms of their binding affinity to an antigen. The affinity of an antibody for an antigen can be determined experimentally using methods known in the art. The term “high affinity” for an antibody typically refers to an equilibrium association constant (K_{aff}) of at least about 1×10⁷ liters/mole, or at least about 1×10⁸ liters/mole, or at least about 1×10⁹ liters/mole, or at least about 1×10¹⁰ liters/mole, or at least about 1×10¹¹ liters/mole, or at least about 1×10¹² liters/mole, or at least about 1×10¹³ liters/mole, or at least about 1×10¹⁴ liters/mole or greater. K_{D}, the equilibrium dissociation constant, can also be used to describe antibody affinity and is the inverse of K_{aff}.

BclA3 spore antigens and antibodies described herein are typically combined with a pharmaceutically acceptable carrier or excipient to form a pharmaceutical composition. Pharmaceutically acceptable carriers can include a physiologically acceptable compound that acts to, e.g., stabilize, or increase or decrease the absorption or clearance rate of a pharmaceutical composition. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of glycopeptides, or excipients or other stabilizers and/or buffers. Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. Detergents can also be used to stabilize or to increase or decrease the absorption of the pharmaceutical composition, including liposomal carriers. Pharmaceutically acceptable carriers and formulations are known to the skilled artisan and are described in detail in the scientific and patent literature, see e.g., the latest edition of Remington's Pharmaceutical Science, Mack Publishing Company, Easton, Pa. (“Remington's”). One skilled in the art would appreciate that the choice of a pharmaceutically acceptable carrier including a physiologically acceptable compound depends, for example, on the route of administration of the composition, antigen, or antibody of the invention, and on its particular physio-chemical characteristics.

Compositions and vaccines of the present invention may be administered by any suitable means, for example, orally, such as in the form of pills, tablets, capsules, granules or powders; sublingually; buccally; parenterally, such as by subcutaneous, intravenous, intramuscular, intraperitoneal or intrastemal injection or using infusion techniques (e.g., as sterile injectable aqueous or non-aqueous solutions or suspensions); nasally, such as by inhalation spray, aerosol, mist, or nebulizer; topically, such as in the form of a cream, ointment, salve, powder, or gel; transdermally, such as in the form of a patch; transmucosally; or rectally, such as in the form of suppositories. The present compositions may also be administered in a form suitable for immediate release or extended release. Immediate release or extended release may be achieved by the use of suitable pharmaceutical compositions, or, particularly in the case of extended release, by the use of devices such as subcutaneous implants or osmotic pumps.

It is often advantageous to formulate oral or parenteral compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic or immunogenic effect in association with the required pharmaceutical carrier. Compositions of peptides, glycans or antibodies, when administered orally, can be protected from digestion, using methods known in the art (see, e.g., Fix, Pharm Res. 13: 1760-1764, 1996; Samanen, J. Pharm. Pharmacol. 48: 119-135, 1996).

In an embodiment, a composition or vaccine is prepared as an injectable, either as a liquid solution or suspension, or as a solid form which is suitable for solution or suspension in a liquid vehicle prior to injection. In another embodiment, a composition or vaccine is prepared in solid form, emulsified or encapsulated in a liposome vehicle or other particulate carrier used for sustained delivery. For example, a vaccine can be in the form of an oil emulsion, a water in oil emulsion, a water-in-oil-in-water emulsion, a site-specific emulsion, a long-residence emulsion, a sticky emulsion, a microemulsion, a nanoemulsion, a liposome, a microparticle, a microsphere, a nanosphere, or a nanoparticle. A vaccine may include a swellable polymer such as a hydrogel, a resorbable polymer such as collagen, or certain polyacids or polyesters such as those used to make resorbable sutures, that allow for sustained release of a vaccine.

In some embodiments, compositions provided herein include one or more additional therapeutic or prophylactic agents for CDI. For example, a composition may contain a second agent for preventing or treating C. difficile infection. Examples of such second agents include, without limitation, antibiotics (such as metronidazole and vancomycin) and antibodies (such as antibodies that bind to toxin A, toxin B, lipoteichoic acid (LTA), PS-I, PS-II, a C. difficile vegetative cell surface protein, or a C. difficile spore cell surface protein such as BclA1, BclA2, Alr, SIpA paralogue, SIpA HMW, CD1021, lunH, Fe—Mn—SOD, and FliD).

In alternative embodiments, compositions of the present invention may be employed alone, or in combination with other suitable agents useful in the prevention or treatment of CDI. In some embodiments compositions of the present invention are administered concomitantly with a second composition comprising a second therapeutic or prophylactic agent for CDI.

As used herein, a “therapeutically effective amount” or “an effective amount” refers to an amount of a composition, vaccine, antigen, or antibody that is sufficient to prevent or treat CDI, to alleviate (e.g., mitigate, decrease, reduce) at least one of the symptoms associated with CDI, and/or to induce an immune response to C. difficile, such that benefit to the subject is provided. The effective amount of a composition, vaccine, antigen, or antibody may be determined by one of ordinary skill in the art. Exemplary dosage amounts for an adult human include, without limitation, from about 0.1 to 500 mg/kg of body weight of antigen or antibody per day, which may be administered in a single dose or in the form of individual divided doses, such as from 1 to 5 times per day, or weekly, or bi-weekly.

In some embodiments, an effective amount of a composition comprising a protein contains about 0.05 to about 1500 pg protein, about 10 to about 1000 pg protein, about 30 to about 500 μg, or about 40 to about 300 pg protein, or any integer between those values. For example, a protein may be administered to a subject at a dose of about 0.1 μg to about 200 mg, e.g., from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, or from about 1 mg to about 2 mg, with optional boosters given at, for example, 1 week, 2 weeks, 3 weeks, 4 weeks, two months, three months, 6 months and/or a year later.

In some embodiments, an effective amount of an antibody composition for passive immunization ranges from about 0.001 to about 30 mg/kg body weight, for example, about 0.01 to about 25 mg/kg body weight, about 0.1 to about 20 mg/kg body weight, about 1 to about 10 mg/kg, or about 10 mg/kg to about 20 mg/kg.

A composition, vaccine, antigen or antibody may also be administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid). For prophylactic purposes, the amount of peptide in each dose is selected as an amount which induces an immunoprotective response without significant adverse side effects in a typical vaccine. Following an initial vaccination, subjects may receive one or several booster immunisations adequately spaced.

It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion and clearance, drug combinations, and severity of the particular condition.

### SgtA Glycosyltransferase

There is also reported herein the identification of a SgtA glycosyltransferase. Insertional inactivation of the glycosyltransferase gene, sgtA (CD3350/CDR3194), provided direct evidence for a role of the glycosyltransferase enzyme in spore surface β-O linked GlcNAc reactivity, as well as in the production of glycosylated BclA3. The sgtA gene is thus linked to production of C. difficile spore glycans and BclA3 spore antigens.

There is provided herein a SgtA glycosyltransferase for use in preparing C. difficile spore antigens as described herein. In an embodiment, SgtA glycosyltransferase is used for production of a BclA3 glycoprotein, glycopeptide or glycan, e.g., in vitro or in a recombinant expression system. In an embodiment, SgtA glycosyltransferase is used for recombinant glycan production. SgtA glycosyltransferase may be prepared using known techniques, e.g., it may be expressed recombinantly and then isolated or purified, or used in an extract from an expression system or cell line.

### Spore Diagnostics and Detection

There are provided methods of diagnosing CDI based on detecting the presence of C. difficile in a subject using a BclA3 spore antigen as described herein. Known methods of detecting bacterial antigens in a sample from a subject may be used to detect the presence of a BclA3 glycoprotein, glycopeptide, or glycan, the presence of the BclA3 glycoprotein, glycopeptide, or glycan being indicative of the presence of C. difficile in the subject. In an embodiment, the presence of the BclA3 glycoprotein, glycopeptide, or glycan in a sample from a subject is indicative of the presence of C. difficile spores in the subject.

A sample may be, for example, a stool sample, including without limitation a liquid stool sample, a solid stool sample, a rectal swab, etc. Antigens may be detected using a variety of common techniques in the art, such as without limitation detection using an antibody reagent specific for a BclA3 antigen, e.g., using an enzyme immunoassay. Alternatively, a nucleic acid amplification test such as PCR may be used to detect the C. difficile BclA3 gene in a sample from a subject.

In one embodiment, there is provided use of the isolated BclA3 glycoprotein or glycopeptide according to any one of claims 1 to 10 or the isolated BclA3 glycan according to any one of claims 11 to 16 for detecting the presence of C. difficile in a subject. In another embodiment, there is provided use of the antibody or fragment of any one of claims 39 to 51 for detecting the presence of C. difficile in a subject. In yet another embodiment, there is provided a method of detecting the presence of C. difficile in a subject comprising: obtaining a stool sample from the subject, and assaying the stool sample for the presence of a BclA3 glycoprotein, glycopeptide, and/or glycan thereof, wherein the presence of the BclA3 glycoprotein, glycopeptide, and/or glycan thereof in the stool sample indicates the presence of C. difficile in the subject.

In some embodiments, methods provided herein are used to detect the presence of C. difficile spores in a subject. Diagnostic methods provided herein may thus, in some embodiments, provide an advantage over current diagnostic methods for CDI that rely on the detection of C. difficile toxin; as C. difficile toxin is produced by vegetative cells and not spores, such methods are not highly effective at detecting the presence of C. difficile spores in patients, e.g., in patients at risk of recurrence of CDI. In an embodiment, methods provided herein are used to diagnose patients at risk of recurrence of CDI based on detecting the presence of C. difficile spores in a subject using a BclA3 spore antigen as described herein

### Kits

Kits are provided for preventing or treating CDI, comprising one or more BclA3 spore antigen, antibody, composition, and/or vaccine as described herein. Instructions for use or for carrying out the methods described herein may also be provided in a kit. A kit may further include additional reagents, solvents, buffers, adjuvants, etc., required for carrying out the methods described herein.

Also provided are kits for diagnosing CDI in a subject or for determining that a subject is at risk of recurrence of CDI comprising reagents for detecting the presence of one or more of a BclA3 glycoprotein, glycopeptide, and glycan in a subject. Instructions for use or for carrying out the diagnostic methods described herein may also be provided in a kit, as well as additional reagents, solvents, buffers, etc., required for carrying out the methods described herein.

As used herein, the singular forms “a”, “an” and “the” include plural references unless the content clearly dictates otherwise.

As used herein, the term “about” refers to a value that is within the limits of error of experimental measurement or determination. For example, two values which are about 5%, about 10%, about 15%, or about 20% apart from each other, after correcting for standard error, may be considered to be “about the same” or “similar”. In some embodiments, “about” refers to a variation of ±20%, ±10%, or ±5% from the specified value, as appropriate to perform the disclosed methods or to describe the disclosed compositions and methods, as will be understood by the person skilled in the art.

The technology described herein is not meant to be limited to particular methods, reagents, compounds, compositions or biological systems, which can, of course, vary. It should also be understood that terminology used herein is for the purpose of describing particular aspects only, and is not intended to be limiting.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the present technology.

### Example 1

### Bioinformatic Identification of BclA and BclB Homologs in Strains of C. difficile

In contrast to spores of C. difficile, the spores of another important toxin producing, Gram positive pathogen, Bacillus anthracis have been extensively characterised. These spores are enclosed by an exosporangial layer which is composed of a number of different proteins, and which includes an outermost hair-like nap layer. The filaments of the nap layer are primarily composed of a highly immunogenic collagen-like protein BclA as well as a second exosporangial protein, BclB. Both BclA and BclB have been well characterised and shown to be glycosylated with an O-linked pentasaccharide which contains the novel terminal sugar, 2-O-methyl-4-(3-hydroxy-3-methylbutamido)-4,6-dideoxy-D-glucopyranose (also referred to as anthrose).

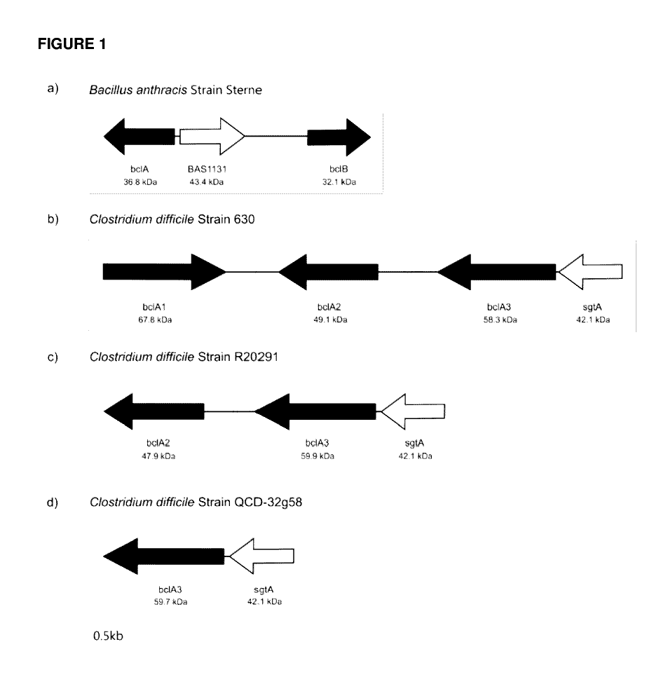

The Gram positive spore forming bacterium Bacillus anthracis thus elaborates two glycosylated spore surface proteins, denoted BclA (BAS1130, YP_027402 in strain Sterne) and BclB (BAS2281, YP_028542 in strain Sterne) for Bacillus collagen-like protein (FIG. 1a). Homologs of BclA have also been found within the genome sequences of Bacillus cereus, and Bacillus thuringiensis (Todd, S. J. et al., J. Bacteriology 185:3373-3378, 2003). BLAST searches of the BclA and BclB sequences against genome sequences of three C. difficile strains revealed Bcl protein homologs. C. difficile 630, the first strain to have a completed genome sequence, had three ORFs with homology to BclA and BclB. These were found in different regions of the C. difficile 630 genome, as shown in FIG. 1b. Percentage homology and E-values of significance are shown in Table 2. The ORF CD3349 was annotated as BclA3 (YP_001089866), despite showing greater homology to BclB, therefore we will refer to this gene as BclA3, to remain consistant with the genome annotation. FIG. 1b also shows C. difficile 630 CD3349 (BclA3, YP001089866) to be located 66 bp downstream of a putative glycosyltransferase (CD3350, YP_001089867). The glycosyltransferase gene has high homology to a B. anthracis glycosyltransferase (BAS 1131, YP_027403) that is likely responsible for transfer of carbohydrate components to exosporangial proteins in this species. The proximity of the genes and high homology with genes of known function within B. anthracis provided early suggestions that C. difficile exosporangial proteins may be glycosylated.

We next searched the genomes of C. difficile strains QCD-32g58 and R20291. Within the strain QCD-32g58 genome, only a single ORF had significant homology to the Bcl proteins; CdifQ_040500019311 (WP_009891815), showed 62% homology to BclA and 50% homology to BclB of B. anthracis. A BLAST search of the glycosyltransferase gene BAS 1131 against the QCD-32g58 genome sequence showed a homolog: a putative glycosyltransferase (CdifQ_040500019316, WP_009891817) 78 bp upstream of the putative exosporium glycoprotein gene (CdifQ_040500019311). The genome of strain R20291 contained two bcl gene homologs, with the translated product of CDR20291_3090 (BclA2, YP_003219565), showing 64% homology to the B. anthracis BclB and CDR20291_3193 (BclA3, YP_003219669) showing 56% homology to the B. anthracis BclA. In addition, CDR20291_3194 (YP_003219670) which lies 78 bp downstream of BclA3 showed 46% identity with the B. anthracis glycosyltransferase (Table 2).

In all three strains, the Bcl protein homologs have only short, trypsin susceptible regions (as predicted by amino acid sequence) at the N and C-termini. The central region of the Bcl protein homologs is comprised of approximately 40 kDa of collagen like repeats with no predicted trypsin cleavage site. The three C. difficile glycosyltransferase homologs have identical sequences apart from a single conserved amino acid substitution at the third amino acid in the sequence.

TABLE 2
BLAST search of Bacillus anthracis exosporangial glycoproteins (BclA & BclB)
translated gene sequences against selected Clostidium difficile translated genomes. Shown are
the percentage sequence identity and expect values (E-value in brackets).

| | | | | | | C. difficile |
|---|---|---|---|---|---|---|
| | C. difficile | | | C. difficile | | Strain QCD-32g58 |
| | Strain 630 | | | Strain R20291 | | Putative |
| B. anthracis | Exosporium | Exosporium | Exosporium | Exosporium | Exosporium | exosporium |
| Strain | glycoprotein | glycoprotein | glycoprotein | glycoprotein | glycoprotein | glycoprotein |
| Sterne | BclA1 | BclA2 | BclA3 | BclA2 | BclA3 | BclA3 |
| BclA | 60% | 57% | 49% | 56% | 56% | 62% |
| | (2e−68) | (4e−49) | (1e−41) | (1e−52) | (7e−32) | (7e−41) |
| BclB | 69% | 62% | 50% | 64% | 50% | 50% |
| | (1e−38) | (2e−34) | (1e−35) | (8e−37) | (1e−35) | (3e−35) |

| | | | Glycosyl |
|---|---|---|---|
| | Glycosyl transferase | Glycosyl transferase | transferase |
| Glycosyl | 47% | 46% | 46% |
| transferase | (7e−114) | (4e−113) | (4e−113) |

### Example 2

### Characterisation of C. difficile Exosporangial Surface Extraction

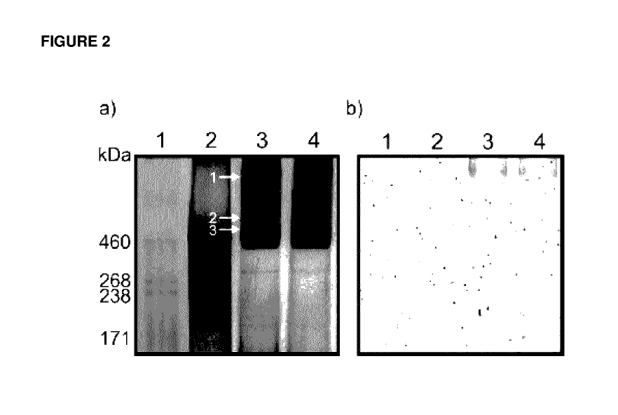

It has been demonstrated previously that C. difficile spores possess an exosporangial layer which surrounds the spore coat and this layer has been shown to be structurally variable amongst isolates. Various treatments of spores have been utilised to remove this layer, allowing the characterisation of the underlying spore coat, however the structural components of the exosporangial layer have not previously been characterised. Here we focused on identifying and characterising spore surface associated protein components. Using a detergent based extraction, surface associated components were removed from spore preparations which had not been extensively water washed or treated with enzymes/sonication to facilitate retention of surface structures. Endospores of strains 630, QCD-32g58 and R20291 were incubated in detergent solutions to extract the spore surface proteins and then intact spores were removed by centrifugation. The protein containing supernatants were resolved by NuPAGE 3-8% gradient gel. The high molecular weight region of the gel showed diffuse banding patterns reactive with both silver stain (FIG. 2a) and glycostain (FIG. 2b), suggesting high molecular weight complexes containing glycoproteins. Proteinase K digestion of spores had only a marginal effect on the migration of this material suggesting a more complex composition. A distinct pattern of staining was obtained for R20291 and QCD32g58 spore extracts when compared to 630 in this region. Glycostaining revealed a reactive high molecular weight band in R20291 and QCD32g58 extracts only.

Initially, extraction of all gel bands of molecular weight <160 kDa was performed and each band was digested with either trypsin or proteinase K and analysed by nLC-MS/MS. No BclA protein identification was made from these analyses. Subsequently, the high molecular weight region (>160 kDa) of each lane was excised in bands, and digested with trypsin or proteinase K. The trypsin digests for all three strains did not result in any protein identifications by nLC-MS/MS. Analysis of the MS/MS spectra from proteinase K digests, however, yielded several spore surface protein identifications, as indicated by the numbered annotations in FIG. 2a, Lane 3 and summarized in Table 3 for R20291, and FIG. 8 and Table 4 for QCD-32g58.

De novo sequencing of the peptide MS/MS spectra from the proteinase K digests of gel bands 1-3 from surface extract of R20291 spores revealed a number of peptides corresponding to the putative exosporium glycoprotein BclA3 (CDR20291_3193). Further inspection of the MS/MS spectra showed peptides with ions that did not correspond to peptide y or b type ions and were characteristic of carbohydrate associated fragment ions. For example, from tandem mass spectrometry analyses of band 1 (FIG. 2a) which migrated to a molecular mass of greater than 600 kDa, the MS/MS spectrum of the putative glycoprotein peptide AGLIGPTGATGV modified with three N-acetyl hexosamine (HexNAc) moieties is shown in FIG. 3a. The glycan modification was observed as sequential neutral losses of 203 Da from the glycopeptide precursor ion in the high m/z region of the MS/MS spectrum. In addition, an intense glycan oxonium ion was observed at m/z 204 which was common to all of the identified glycopeptides. In addition more complex glycosylation patterns were observed in some cases, with intense ions observed in glycopeptide spectra that did not correspond to HexNAc residues nor peptide type y or b fragment ions (for example, oxonium ions corresponding to masses of 486 Da, 372 Da, 374 Da). FIG. 3b shows an MS/MS spectrum of the BclA peptide TGPTGATGADGITGP, modified with three HexNAc moieties and an additional mass of 374 Da. The sequential neutral losses suggest that HexNAc is the linking sugar. This glycan neutral mass was also linked to a putative glycan oxonium ion at m/z 375. Other intense ions were also observed in the low m/z region of this MS/MS spectrum, including putative glycan fragment ions at m/z 300 and 272. The absence of potential N-linked glycosylation sites suggested the glycans to be O-linked through threonine residues within each peptide. Observed sequential neutral losses of 203 Da in the high m/z region of the spectrum and the presence of an intense ion corresponding to the unmodified form of the peptide suggest the glycan is composed of oligosaccharide chains attached to a single amino acid residue in each identified glycopeptide.

Table 3 shows the complete list of surface protein peptides and glycopeptides identified from each of the annotated band numbers indicated in FIG. 2a. The unknown glycans varied in observed mass from 281 to 486 Da and it is possible that these are modified HexNAc moieties. Tandem mass spectrometry analysis of proteinase K digests of bands 2 and 3 showed BclA3 glycopeptides modified with chains of HexNAc moieties, predominantly in trimers. In these cases, only peptides modified with HexNAc moieties were observed at detectable levels.

TABLE 3
nLC-MS/MS analysis of glyco-reactive peptides. High molecular weight gel bands of
R20291 spore surface extracts were digested with proteinase K and the numbering of gel bands
refers to FIG 2. MS/MS spectra were de novo sequenced, and the identified peptides and
observed glycan moieties are indicated

| | Accession | Protein | | Glycan Mass, Da (Monosaccharide |
|---|---|---|---|---|
| Band | number | (MW, kDa) | Peptide sequence | neutral losses, Da) |
| 1 | YP_003219669 | Exosporium | ³⁰⁸AGLIGPTGATGV³¹⁹ | 609 (203-203-203) |
| | (CDR20291_3193) | glycoprotein | ¹⁴⁵TGPTGATGADGITGP¹⁵⁹ | 983 (203-203-203-374) |
| | | BclA3 | ³⁴⁴VGPTGATGA³⁵² (also aa³⁹¹⁻³⁹⁹, | 406 (203-203) |
| | | (59.5 kDa) | aa⁴³⁹⁻⁴⁴⁷ aa⁴⁸⁷⁻⁴⁹⁵) | |
| | | | ¹⁸⁹GLIGPTGATGTPGA²⁰² | 406 (203-203) |
| | | | ²⁷⁸TGATGLIGPTGATGA²⁹² | 1186 (203-203-203-203-374) |
| | | | ²⁷⁸TGATGLIGPTGATGA²⁹² | 1241 (203-203-203-203-429) |
| | | | ²¹²TGIGITGPTGATGA²²⁵ (also aa²⁵⁹⁻²⁷²) | 1184 (203-203-203-203-372) |
| | | | ²¹²TGIGITGPTGA²²² (also aa²⁵⁹⁻²⁶⁹) | 1095 (203-203-203-486) |
| | | | ³⁰⁹GLIGPTGATGVTGA³²² | 1071 (203-203-203-462) |
| | | | ²⁹⁹TGVTGATGAAGLIGP³¹³ | 609 (203-203-203) |
| 2 | YP_003219669 | Exosporium | ²⁷⁸TGATGLIGPTGATGA²⁰² | 1187 (203-203-203-203-375) |
| | (CDR20291_3193) | glycoprotein | ¹⁹¹IGPTGATGTPGATGPTGA²⁰⁸ | 1661 (203-203-203-222-203-203-424) |
| | | BclA3 | ⁴²⁴TGPTGATGPTGADGL⁴³⁸ | 609 (203-203-203) |
| | | (59.5 kDa) | | |
| 3 | YP_003219669 | Exosporium | ¹¹⁹GVTGPTGPTGPTGATGA¹³⁵ | 609 (203-203-203) |
| | (CDR20291_3193) | glycoprotein | ¹⁶⁹GVTGPTGPTGATGV¹⁸² | 609 (203-203-203) |
| | | BclA3 | ³⁴⁴VGPTGATGATGADGL³⁵⁸ | 609 (203-203-203) |
| | | (59.5 kDa) | ³⁵⁹VGPTGPTGATGV³⁷⁰ | 609 (203-203-203) |
| | | | ¹⁹¹IGPTGATGTPGATGPTGA²⁰⁸ | 609 (203-203-203) |
| | | | ³¹¹IGPTGATGVTGADGA³²⁵ | 609 (203-203-203) |
| | | | ⁴³⁹VGPTGATGATGL⁴⁵⁰ | 609 (203-203-203) |
| | | | ³⁹¹VGPTGATGATGADGV⁴⁰⁵ | 609 (203-203-203) |
| | | | ³⁵⁹VGPTGPTGATGV³⁷⁰ | 203 |
| | | | ⁶⁵⁶ATASGLSLVNTVA⁶⁶⁸ | — |

Table 4 shows a list of surface protein peptides and glycopeptides identified from annotated band numbers as indicated in FIG. 8.

TABLE 4
nLC-MS/MS analysis of glyco-reactive peptides. MS/MS spectra were
de novo sequenced, and the identified peptides and observed glycan
moieties are indicated.

| Unique/repeated | | | |
|---|---|---|---|
| sequence | m/z | Peptide Sequence | Glycan mass, Da |
| Unique | 1001.51²⁺ | ¹⁶⁶GVTGPTGPTGATGV¹⁷⁹ | 203-203-203-220 |
| | | (SEQ ID NO: 27) | |
| | 901.42²⁺ | ²⁹³TGPTGVTGATGA³⁰⁴ | 203-203-203-203 |
| | | (SEQ ID NO: 25) | |
| | 811.89²⁺ | ³⁰⁵AG LIGPTGATGV³¹⁶ | 203-203-203 |
| | | (SEQ ID NO: 2) | |
| | 1088.5²⁺ | ³⁰⁵AG LIGPTGATGV³¹⁶ | 203-203-203-552 |
| | | (SEQ ID NO: 2) | |
| | 927.43²⁺ | ³⁰⁸IG PTGATGVTGADGA³²² | 203-203-203 |
| | | (SEQ ID NO: 32) | |
| | 998.48²⁺ | ³⁵⁴GLVGPTGPTGATGV3⁶⁷ | 203-203-203-215 |
| | | (SEQ ID NO: 29) | |
| | 1130.51²⁺ | ⁴⁰³AGPTGATGATGNTGADGA⁴²⁰ | 203-203-203-203 |
| | | (SEQ ID NO: 30) | |
| | 1043.49²⁺ | ⁴²¹TGPTGATGPTGADGL⁴³⁵ | 203-203-203-203 |
| | | (SEQ ID NO: 13) | |
| | 1052.03²⁺ | ⁴²¹TGPTGATGPTGADGL⁴³⁵ | 203-203-203-220 |
| | | (SEQ ID NO: 13) | |
| | 806.4²⁺ | ⁴³⁶VGPTGATGATGL⁴⁴⁷ | 203-203-203 |
| | | (SEQ ID NO: 20) | |
| Repeated | 597.80²⁺ | TGPTGADGL (x4) | 203-203 |
| | | (SEQ ID NO: 28) | |
| | 978.96²⁺ | GVTGPTGPTGATGA (x3) | 203-203-203-203 |
| | | (SEQ ID NO: 26) | |

All of the identified glycopeptides reside within the central collagen like repeating domain of the BclA3 protein. The central collagen-like repeat domains of the putative exosporial proteins contained some non-unique regions, which resulted in the identification of multiple glycopeptides with amino acid sequences that repeat within the protein sequence (for example, TGIGITGPTGA occurs in BclA3 at residues 212-222 and 259-269). One of the identified peptides, which is repeated in BclA3 four times, is also common to both BclA3 and BclA2 (VGPTGATGA). The non-specific cleavage by proteinase K produced a number of glycopeptides with overlapping sequences. Furthermore, multiple glycopeptides were identified that possessed identical peptide sequences but different glycans.

As indicated above, strains R20291 and QCD-32g58 showed similar protein staining patterns for both silver and glycostains and nLC-MS/MS analysis also showed that the Bcl protein of QCD-32g58 was similarily glycosylated predominantly with HexNAC moieties (FIG. 8). nLC-MS/MS analysis of the gel digests from strain 630, which showed significantly different staining patterns in the high molecular weight region of the gel as compared to the other two strains, did not yield any protein or glycoprotein identifications.

### Example 3

### Anti-β-O-GlcNAc Reactivity of C. difficile Spores

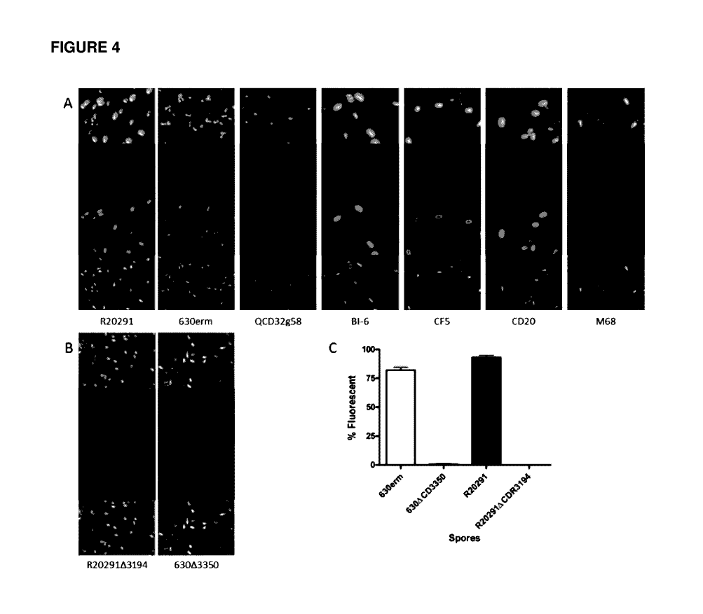

As the most abundant glycan modification observed in the MS analysis of spore surface extracted material was shown to have a mass corresponding to an N acetyl-hexosamine moiety, we next examined the ability of spores to bind to an O-linked N-Acetyl glucosamine (β-O-GlcNAc) antibody. A monoclonal antibody (MAb) which recognises O-GlcNAc in a β-O-glycosidic linkage to both threonine and serine was utilised in immunofluorescence experiments with intact spores from a number of C. difficile clinical isolates (FIG. 4A). This antibody had been used previously to demonstrate presence of β-O-GlcNAc attached to serine and threonine residues of Listeria monocytogenes flagellin (Schirm, M. et al., J. Bacteriology 186: 6721-6727, 2004).

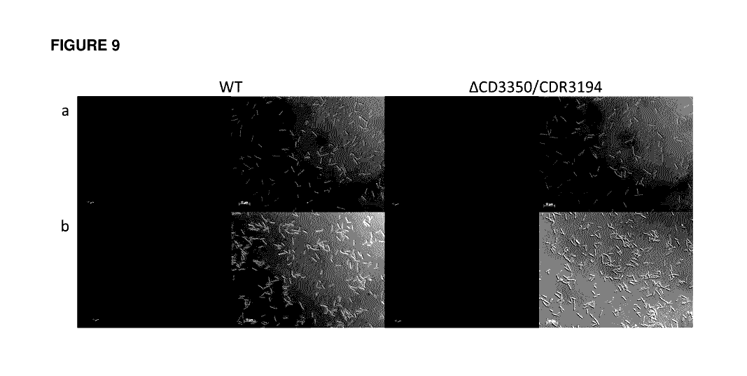

When the β-O GlcNAc antibody was used in immunofluorescence reactions with spores of R20291 and 630Δerm, both spore preparations reacted strongly with the antibody. Interestingly, distinct patterns of reactivity with the spore surface were observed by this immunofluorescence method for each strain (FIG. 4A). With R20291 spores anti-β-O-GlcNAc was uniformly reactive over the entire spore surface, while with strain 630Δerm, GlcNAc reactivity was restricted to the poles of the spores with only limited labelling of the central surface (see arrows marking binding at poles; FIG. 4A). Vegetative cells of both strains showed no reactivity with anti-β-O-GlcNAc (FIG. 9). To confirm the conservation of β-O-GlcNAc on the surface of multiple C. difficile strains, a range of spores from different ribotypes and geographic locations were also tested for anti-β-O-GlcNAc binding. The reactivity pattern observed for R20291 spores was found to be conserved in all strains examined, with the only exception being spores of 630Δerm (FIG. 4A). Any unstained cells in the images were either immature spores or cell debris from the washing process. DAPI binding was observed only with vegetative cells and immature phase dark spores. Phase bright spores were considered mature.

### Example 4

### Characterization of SgtA Glycosyltransferase

RT-PCR of CD3350/bclA3 Gene Locus.

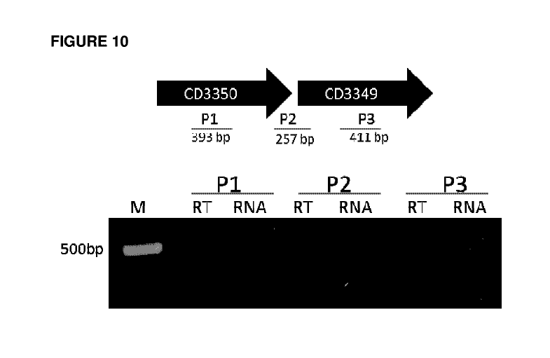

As indicated in FIG. 1, CD3350 (B. anthracis exosporangial glycosyltransferase gene homolog) and BclA3 lie immediately adjacent to each other and are orientated in the same direction on the chromosome in both 630 and R20291 strains. The two genes are separated by only a short intergenic region suggesting they may form a single transcriptional unit. Primers which amplified across this intergenic region were used to determine if the genes were co-transcribed. RNA samples extracted from C. difficile 630 cells were subjected to reverse transcription and an amplification product of 257 bp linking CD3350 and bclA3 was obtained confirming cotranscription of these two genes (FIG. 10). PCRs using the same primers and total RNA that had not undergone reverse transcriptase reaction did not yield any amplification product demonstrating that the RNA was free of contaminating DNA.

Mutagenesis of CD3350/CDR3194 and Spore Characterisation.

We next generated an insertionally inactivated glycosyltransferase mutant in strains 630Δerm and R20291 respectively (ΔCD3350 and ΔCDR3194) by using the ClosTron technology as previously described (Heap, J. T. et al., J. Microbio. methods 80:49-55, 2010). Insertion of the TargeTron Erm resistance marker was confirmed by PCR using primers flanking the gene of interest and with primers specific to the TargeTron Erm resistance marker (data not shown). Vegetative cell growth of both ΔCD3350 and ΔCDR3194 were unchanged when compared to their respective parent strains and motility was unaffected (data not shown). Immunofluorescence of spores, with anti-β-O-GlcNAc antibody, revealed a complete loss of reactivity for both ΔCD3350 and ΔCDR3194 when compared to respective parent strains (FIG. 4B). The percentage of wild type spores compared to mutant phase bright spores reacting with anti-β-O-GlcNAc antibody was quantified microscopically and shown to be 80-95% compared to less than 1% for the respective mutants (FIG. 4C).

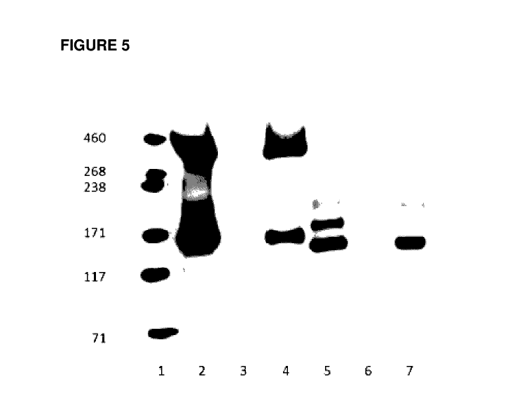

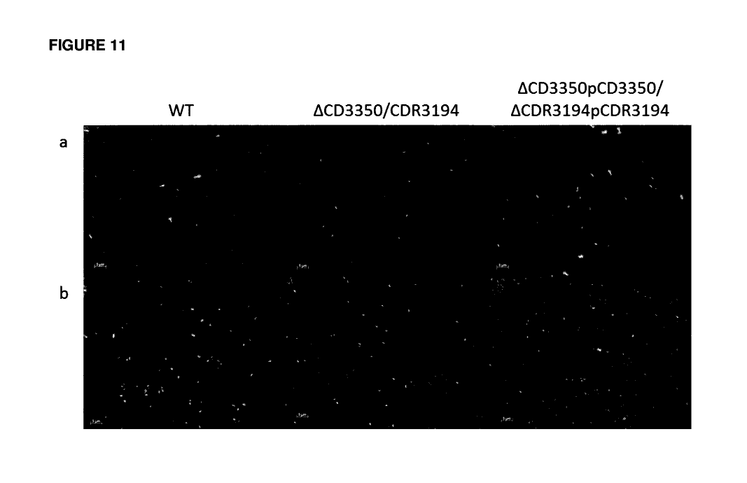

Both ΔCDR3194 and ΔCD3350 strains were complemented with wild type copies of CD3350 using pRPF185 (Fagan, R. P. et al., J. Biol. Chem. 286:27483-27493, 2011) as evidenced by both western blotting and immunofluorescence studies (FIG. 5 and FIG. 11). As can be seen in FIG. 5 lanes 2 and 5, a positive reaction was observed in western blot with spore surface extracts from both R20291 and 630Δerm spores respectively. Spore extracts of R20291 displayed reactivity with the region of gel corresponding to band 4 (approximately 400 kDa) from MS analysis. In addition, a second strongly reactive band migrating at molecular mass of approximately 170 kDa on 3-8% NuPAGE gel was observed, although we were unable to identify peptides from a proteinase K digestion of this region of the gel by MS analysis. For strain 630Δerm, no reactivity was observed in the corresponding higher molecular weight region of the gel and a series of three distinct reactive bands were observed at approx. 170 kDa. All reactivity was lost in CD3350 and CDR3194 mutant strains while the strain specific pattern of reactivity was restored upon complementation (FIG. 5 and FIG. 11). On the basis of these results this gene was named sgtA for spore glycosyltransferase.

Characterisation of ΔsgtA Spore Surface Extract.

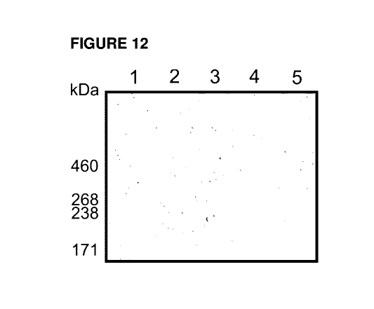

In parallel with the spore surface protein extracts of the wild type strain, spore surface extracts of the R20291ΔsgtA mutant strain were also prepared and analysed by NuPAGE gradient 3-8% Tris acetate gels to resolve high molecular weight material. The protein stained gel shows a diffuse area of staining at 460 kDa and greater, however, the distinct ˜600 kDa band was not observed. Similarly, glycostaining of the same gel showed no detectable reactivity at ˜600 kDa (FIG. 12). In contrast to MS studies of gel bands of R20291 spore surface extracts which identified peptides/glycopeptides in Proteinase K digests, the equivalent region of the NuPAGE gel of the spore surface protein extraction of ΔsgtA did not yield any peptide or glycopeptide identifications. In addition, our analyses of lower molecular weight protein bands from spore surface extracts showed no evidence of unglycosylated BclA3.

Resistance of R20291ΔsgtA Spores.

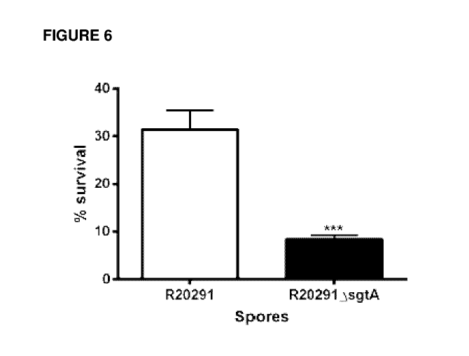

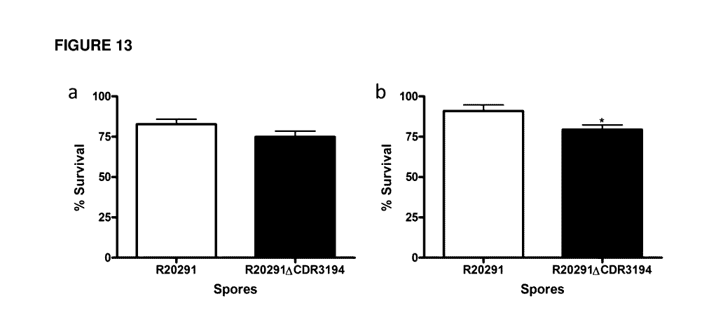

As the more clinically relevant strain and as shown by immunofluorescence to be a more representative strain of C. difficile spore morphology, phenotypic assays were undertaken on R20291 wild type spores compared to ΔsgtA spores. Heat resistance of spores was examined as previously described (Permpoonpattana, P. et al., J. Bacteriology 195:1492-1503, 2013). When incubated at 80° C. for 20 minutes ΔsgtA spores showed significantly lower survival rates than the parent R20291 spores (FIG. 6). Susceptibility of spores to 70% ethanol and 250 μg/ml lysozyme was also examined, but no significant difference was observed between wild type and ΔsgtA spores (FIG. 13).

Role of sgtA in Adherence and Internalisation of Macrophage Cells.

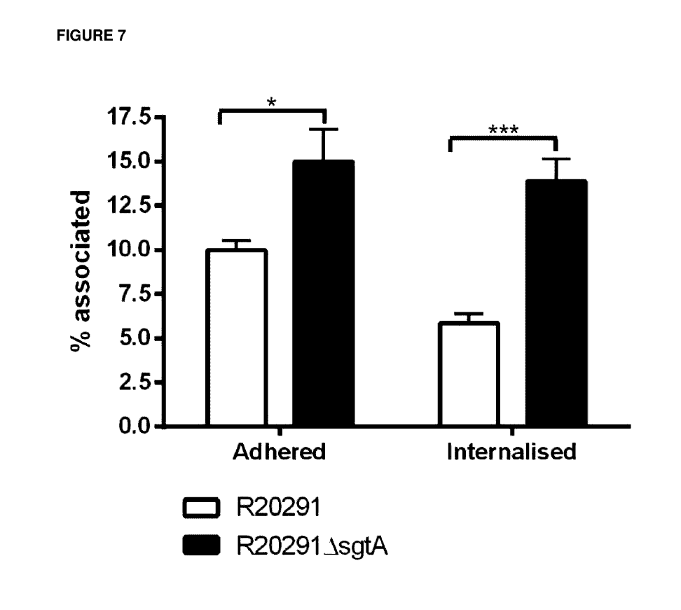

To gain insight into a possible biological role for the SgtA glycosyltransferase we next investigated the ability of spores to adhere to and be internalised by J774A.1 macrophage cell line (ATCC TIB-106). Spores were counted based on association with J774A.1 cells, and counted as adhered if green/red and internalised if red. Spores not associated with cells were ignored as were any remaining vegetative cells based on rod shape. As can be seen in FIG. 7, adherence and internalisation of J774A.1 macrophage cells by C. difficile R20291 spores was affected following inactivation of sgtA gene, with significantly greater numbers of ΔsgtA spores being internalised compared to wild type.

In sum, the studies described above present a characterization of glycoproteins from C. difficile spores and provide direct evidence demonstrating that BclA3 is a glycoprotein which is glycosylated with chains of β O-linked GlcNAc as well as with additional glycans of novel mass. Our nLC-MS/MS analysis identified BclA3 peptides and provides the first evidence that this protein is a glycoprotein. The BclA3 protein of C. difficile is glycosylated with predominantly novel tri- or pentasaccharide oligosaccharides, composed of chains of N-Acetyl hexosamine sugars which in addition, may be capped with novel glycan moieties.

It is clear from glycan component neutral masses that the structural composition of the C. difficile BclA3 glycan is quite distinct to that previously reported for B. anthracis (Daubenspeck, J. M. et al., J. Biol. Chem. 279:30945-30953, 2004). Gel migration characteristics suggested that C. difficile BclA3 monomers from R20291 and QCD-32g58 form a stable, higher molecular weight complex which is resistant to denaturation by heating and detergents.

It is noted that β-O linked GlcNAc reactivity of spores from a number of clinical isolates has demonstrated for the first time the conserved nature of this posttranslational modification on C. difficile spores. Further, insertional inactivation of the glycosyltransferase gene, sgtA (CD3350/CDR3194), provided direct evidence for a role of the glycosyltransferase enzyme in this spore surface β-O linked GlcNAc reactivity, as well as in the production of glycosylated BclA3. Our studies thus link the sgtA gene to a specific spore glycan associated function.

The spores of Gram positive bacterial pathogens have gained considerable attention in recent years. A role for surface-associated bacterial glycans in host interactions has been well documented for many bacterial species. Spores are known to be recalcitrant to proteolytic digestion and structural characterization. The studies described herein showed that spores of a second important Gram positive pathogen, C. difficile, also carry novel glycoproteins on surface associated structures, and that glycans on the spore surface impart resistance of spores to heat treatment as well as appear to play a role in macrophage interactions.

### Materials and Methods

Bacterial Strains and Growth Conditions.

C. difficile strains used in this study are listed in Table 5. Initial experiments were carried out using strains 630Δerm and R20291. Comparisons with other C. difficile strains from a variety of ribotypes (QCD-32g58, BI-6, CD20, CF5, and M68) revealed R20291 to be the more representative strain. R20291 is also a more clinically relevant strain, and a better spore former than strain 630. For these reasons, later experiments, particularly the biological assays, were focused on R20291 spores. All strains were routinely grown under anaerobic conditions on brain heart infusion agar medium (BD, Sparks, MD) supplemented with 5 g/litre yeast extract, 1.2 g/litre NaCl, 0.5 g litre cysteine HCl, 5 mg/litre hemin, 1 mg/litre vitamin K, and 1 mg/litre resazurin (BHIS). Erythromycin (2.5 μg/ml) and thiamphenicol (15 μg/ml) were added as required for growth of mutant and complemented mutant strains.

TABLE 5
C. difficile strains used in this study.

| Strain | Characteristics | Source |
|---|---|---|
| 630Δerm | Ribotype O12 | (*) |
| | | Minton, |
| | | University of |
| | | Nottingham |
| R20291 | Ribotype O27 | (**) Wren, |
| | | LSHTM |
| 630ΔCD3350 | 630Δerm CD3350::erm | This study |
| R20291ΔCDR3194 | R20291 CDR3194::erm | This study |
| 630ΔCD3350p3350 | 630Δerm ΔCD3350 | This study |
| | pRPF185-CD3350 | |
| R20291ΔCDR3194p3350 | R20291ΔCDR3194 | This study |
| | pRPF185-CD3350 | |
| QCD-32g58 | Ribotype 027 | (***): |
| | | Dascal, |
| | | Montreal. |
| BI-6 | Ribotype 0176 | Wren, LSHTM |
| CD20 | Ribotype 023 | Wren, LSHTM |
| CF5 | Riobtype 017 | (§) |
| | | Wren, LSHTM |
| M68 | Ribotype 017 | (§) |
| | | Wren, LSHTM |

(*) Hussain, H. A. et al., J. Med. Micro. 54: 137-141, 2005.
(**) Stabler, R. A. et al., Genome biology 10: R102, 2009.
(***): Forgetta, V. et al., J. Clin. Microbio. 49: 2230-2238, 2011.
(§) He, M. et al., Proc. Natl. Acad. Sci. 107: 7527-7532, 2010.

Mass Spectrometry (MS) Analysis of Spores.

Spores were harvested from BHIS agar plates into PBS, following 7 day incubation under anaerobic conditions, heat treated at 56° C. for 15 minutes, collected by centrifugation (500 g, 30 min) and washed once in PBS. Then cfu/ml was determined by serial dilution and plating on BHI containing 0.1% sodium taurocholate (Sigma-Aldrich, Oakville, Canada) (BHI-ST). Approximately 5×10⁹ spores were resuspended in 200 μl of extraction buffer (2.4 ml 1 M Tris pH 6.8, 0.8 g ASB-14, 4 ml 100% glycerol, 1% DTT, 3.8 ml ddH₂O) and were left for 30 minutes at room temperature. Spores were removed by centrifugation and soluble material was collected for analysis.

Protein containing endospore surface extractions were separated using 3-8% NuPage Novex Tris-Acetate minigels following the manufacturer's instructions (Invitrogen, Life Technologies). High-molecular-mass ‘Hi-mark’ (31 to 500 kDa) were used as markers. The gel was stained using Emerald-Q glycostain, as per the manufacturer's instructions (Invitrogen, Life Technologies) and subsquently with non-fixing silver stain (Blum, H. et al., Electrophoresis 8:93-99, 1987). Protein bands were excised, reduced for 1 hour with 10 mM DTT at 56° C., and alkylated for 1 hour with 55 mM iodoacetamide in the dark (Gharandaghi, F. et al., Electrophoresis 20:601-605, 1999) prior to digestion with trypsin as described previously (Fulton, K. M. et al., IJMM 301:591-601, 2011) or with proteinase K. Proteinase K digests were carried out using 100 μg/ml of enzyme in 50 mM ammonium bicarbonate for 15-40 hours. The resulting peptides were analyzed by nanoliquid chromatography coupled to tandem MS (nLC-MS/MS) using electrospray ionization (ESI) as the ion source as recently described (Fulton, K. M. et al., IJMM 301:591-601, 2011). Briefly, peptides were analyzed by nanoflow reversed-phase liquid chromatography (RPLC) coupled to MS using ESI (nanoRPLC-ESI-MS) using a nanoAcquity UltraPerformance LC (UPLC) system coupled to a Q-TOF Ultima hybrid quadrupole-TOF mass spectrometer (Waters, Milford, Mass.). The peptides were first loaded onto a 180 μm inner diameter (ID) by 20 mm 5 μm symmetry C18 trap column (Waters, Milford, Mass.) and then eluted to a 100 μm ID by 10 cm 1.7 μm BEH130C18 column (Waters, Milford, Mass.) using a linear gradient from 1% to 45% solvent B (ACN plus 0.1% formic acid) in 18 min, 45% to 85% solvent B for 3 min, 85% to 1% solvent B over 1 min. Solvent A was 0.1% formic acid in HPLC grade water. The peak list files of MS/MS spectra from tryptic digests were searched against the NCBI database using the MASCOT search engine (Version 2.3.0 Matrix Science, London, United Kingdom). A mass tolerance for precursor ions of 0.8 Da was used for precursor and fragment ions. Ion scores of 30 and above indicated identity. In addition, all spectral matches were verified manually. Unmatched MS/MS spectra and all MS/MS spectra from proteinase K digests were examined manually to determine the sequences of peptide y and b type ions.

Construction of CD3350/CDR3194 insertional mutants and complemented mutants.

The target site was identified for CD3350 gene from C. difficile 630 using the Targetron gene knockout system (Sigma Aldrich) and was used to design a 45 bp retargeting sequence for the gene. A derivative of plasmid pMTL007C-E2 carrying the retargeting sequence was obtained from DNA2.0 (Menlo Park, Ca) and used to generate mutants in strains 630Δerm and R20291 according to Heap et al. (Heap, J. T. et al., Methods in molecular biology 646:165-182, 2010; Heap, J. T. et al., J. Microbio. methods 80:49-55, 2010). A minimum of two Erm resistant transconjugants for each strain were checked by PCR using the ErmRAM primers to verify splicing of the group I intron following integration and also using flanking primers for the CD3350 gene to verify disruption of CD3350/CDR3194 gene by the erm cassette.

Each of the CD3350/CDR3194 glycosyltransferase mutant strains were complemented with a wild type copy of the C. difficile CD3350 gene using plasmid pRPF185 (Fagan, R. P. et al., J. Biol. Chem. 286:27483-27493, 2011). The entire coding sequence of the gene including the Shine Dalgarno sequence was cloned under the control of the inducible Ptet promoter. Plasmids were transferred to ΔCD3350/ΔCDR3194 mutant strains via conjugation and gene expression induced by plating onto BHIS agar containing anhydrous tetracycline at 500 ng/ml after growth to mid-late log phase in BHIS broth.

Western Blotting.

Spore samples were harvested at 72 h and resuspended to 1×10⁷ spores/100 μl in 1× Laemmli loading buffer and heated to 95° C. for 5 min. Spore extracts were separated on 3-8% NuPage Novex Tris-Acetate minigels and blotted onto PVDF. The membrane was probed with 1:5000 dilution of anti-β-O-GlcNAc (Covance, Montreal, Canada) in PBS-0.1% Tween 20 (PBS-T). Reactivity was detected with anti-mouse IgM HRP conjugate (Sigma Aldrich, Oakville, Canada) secondary antibody at 1:10000 dilution in PBS-T. Blots were imaged with ECL Prime western blotting detection kit (GE Healthcare, Baie D'Urfe, QC, Canada) according to manufacturer's instructions, followed by exposure to X-ray film.

Transcription of CD3350 and BclA3 Genes by RT-PCR.

To determine if the CD3350 and bclA3 genes are cotranscribed, reverse transcriptase PCR (RT-PCR) was performed using primers designed to amplify across the intergenic region between the two genes from C. difficile 630. RNA template was extracted from broth grown cells (4 h) using a Trizol extraction procedure (Aubry, A. et al., Infection and immunity 80:3521-3532, 2012). All RNA samples were treated with RNase free DNase (Thermo Scientific) to remove contaminating DNA. RNA was quantified and 30 ng was used for each RT-PCR using Sensi-script RT kit (Qiagen) and PCR amplification using TopTaq Master. In addition PCR amplifications were performed with the same primers using genomic DNA to verify amplicon size and specificity of primer pairs. Control PCR reactions of RNA without reverse transcriptase confirmed absence of contaminating DNA in samples.

Spore Production for Biological Testing.

For production of mature spores, plates were incubated for 7 days in an anaerobic incubator (Don Whitely Scientific, UK) on BHI at 37° C. Spores were harvested from agar and heat treated at 60° C. for 20 minutes. To purify spores, samples were washed ×10 in H₂O and cfu/ml determined by serial dilution and plating on BHI-ST.

Immunofluorescence.

Spores at 1×10⁸/ml were air dried and heat fixed onto glass coverslips (VWR). The spores were blocked with 5% milk PBS for 30 minutes at room temperature, then incubated with 1:100 dilution in PBS of β-O-GlcNAc monoclonal antibody (Covance) for 45 minutes at room temperature. Coverslips were washed with PBS-T, then incubated with 1:100 dilution in PBS of anti-mouse IgG+IgM FITC conjugate (Caltag, Burlingame, Calif.) for 45 minutes at room temperature in dark. Coverslips were washed with PBS-T then mounted with Vectashield+DAPI (Vector Laboratories, Burlingame, Calif.) onto slides. Slides were examined with Axioplan 200M (Zeiss), with multiple fields of view observed. The experiment was performed in duplicate on at least three independent occasions. For quantification of GlcNAc reactivity to spores, slides were prepared as stated above using 7 day H₂O washed spores, then examined by microscopy. Using an Axioplan200 M microscope (Zeiss), at least 8 fields of view were examined per slide, with three replicate slides per sample. At least 100 spores per slide were counted for anti-β-O-GlcNAc binding to phase bright spores. This was performed on at least three occasions to enumerate the percentage of fully mature spores that could be bound with anti-β-O-GlcNAc.

Spore Heat Resistance Assay.

Heat resistance of C. difficile spores was determined as previously described (Permpoonpattana, P. et al., J. Bacteriology 195:1492-1503, 2013). Briefly spores of R20291 and CDR3194 mutant strains were resuspended in 5 ml of PBS at 1×10⁶/ml with starting inocula numbers confirmed by serial dilution and plating on BHI-ST, incubated anaerobically for 24 hours at 37° C. 1 ml aliquots of spores were heated to 80° C. for 20 minutes in a water bath, then plated on BHI-ST and incubated anaerobically for 24 hours at 37° C. to determine cfu/ml. Percentage survival was determined by comparing pre and post heat treatment cfu/ml. The experiment was performed in triplicate on at least three independent occasions.

Spore Lysozyme Resistance Assay.

Spores of R20291 and CDR3194 mutant strains were diluted to 1×10⁶/ml in 5 ml PBS with starting inocula numbers confirmed by serial dilution as described above. Lysozyme was added to a final concentration of 250 μg/ml and 1 ml samples were incubated for 1 hour at 37° C. Cfu/ml was determined by serial dilution and plating on BHI-ST. Percentage survival was determined by comparing pre and post lysozyme treatment cfu/ml. Experiment performed in triplicate on three independent occasions.

Spore Ethanol Resistance Assay.

Spores of R20291 and CDR3194 mutant strains were diluted to 1×10⁶/ml in 5 ml 70% ethanol. Time point 0 cfu/ml was confirmed by serial dilution and plating on BHI-ST as described above. 1 ml aliquots were incubated at room temperature for 20 minutes, then plated on BHI-ST and incubated anaerobically for 24 hours at 37° C. to determine cfu/ml. Percentage survival was determined by comparing pre and post ethanol treatment cfu/ml. Experiment performed in triplicate on three independent occasions.

Macrophage Assay.

J774A.1 macrophages were cultured at 5×10⁵ cells/well on coverslips, in 24 well plate in 1 ml RPMI media supplemented with 10% FBS (R10), at 5% CO₂ 37° C. for 24 hours. Spores were diluted to 5×10⁶/ml (MOI 10:1) in R10 and inocula calculated by dilution series and plating on BHI-ST. J774A.1 cells were washed with PBS then 1 ml spores added to four replicate wells. Plate was incubated for 30 minutes at 37° C. 5% CO₂, then wells were washed with PBS, before cells were fixed with 250 μl 4% formaldehyde for 15 minutes at room temperature. Cells were washed with PBS then incubated with a rabbit anti-C. difficile polyclonal antisera (CD3) at 1:100 dilution in PBS for 45 minutes at room temperature. Coverslips were washed with PBS, then incubated with anti-rabbit Alexafluor 488 (Invitrogen) at 1:1000 dilution PBS for 45 minutes, at room temperature. Coverslips were washed with PBS, then cells were permeabilised with 0.1% Triton-PBS for 15 minutes, at room temperature. Coverslips were washed with PBS then incubated for 45 minutes at room temperature in 1:100 dilution of CD3 antisera in PBS. Coverslips were washed in PBS, then incubated in 1:1000 dilution of anti-rabbit Alexafluor 594 (Invitrogen) for 45 minutes, at room temperature. Coverslips were washed with PBS, then mounted onto slides with Vectashield+DAPI (Vector Laboratories) and sealed with nail polish. Slides were examined with Axioplan 200M microscope (Zeiss). Three coverslips per strain with 50 J774A.1 cells per coverslip were counted utilising z stack images to gain a 3D representation of the cell. Adhesion and internalisation were quantified by counting adhered (red/green) and internalised (red) spores and calculating percentage adhesion or internalised per J774A.1 cell based on known MOI. Assay was performed in triplicate on three independent occasions.

Statistical Analysis.

Student's t-test with Welch's correction was used for pairwise comparisons.

### Example 6

### Polyclonal Immune Serum Production to Spore Surface

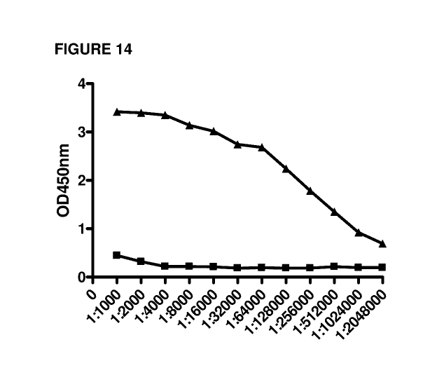

Further work was completed to investigate the immunogenicity of spore BclA3 glycoprotein on the spore surface. Formalin killed spores from C. difficile strain R20291 were used to immunise New Zealand white rabbit to produce a high titre polyclonal antiserum (CD5). Immunization included sub-cutaneous immunisation with 1×10⁸ spores, and two boosts all in incomplete Freund's adjuvant (FA). This immune serum was tested against viable R20291 spores by ELISA. The results showed that rabbit polyclonal serum produced after immunization with formalin killed cells clearly recognised R20291 spores when coated on an ELISA plate (FIG. 14). Spores were immunogenic upon subcutaneous immunisation with incomplete Freund's adjuvant.

Spore Surface Antigens.

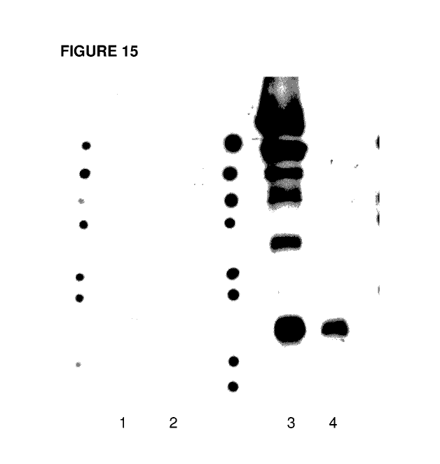

Next, the reactivity of this polyclonal immune serum with spore surface extracts was examined by western blotting. Spores (5×10⁷) of either the R20291 parent strain or R20291::sgtA strain were resuspended in 100 μl of SDS-PAGE solubilisation buffer and heated to 100° C. for 20 minutes. Insoluble material was removed by centrifugation in Eppendorf centrifuge for 15 min and spore surface extract analysed by SDS-PAGE. Western blotting with CD5 antiserum revealed a number of reactive bands in the extract from R20291 parent strain (FIG. 15). In contrast, spore surface extracts from R20291::sgtA had only limited reactivity with the CD5 serum indicating loss of immune reactive material as a consequence of sgtA inactivation. BclA3 glycoprotein was previously shown to migrate to regions of the gel corresponding to the top two reactive bands in the immunoblot. In conclusion, immunoblotting using the CD5 antiserum identified immunoreactive bands in spore extracts by Western blotting, and identified BclA3 as a key immunogen. Further, our results indicate that insertional inactivation of glycosyltransferase resulted in almost complete loss of immunoreactivity, demonstrating immunogenicity and significance of glycan structure.

Although this invention is described in detail with reference to preferred embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

What is claimed is:

1. An isolated C. difficile spore BclA3 glycoprotein or a glycopeptide thereof.

2. The isolated BclA3 glycoprotein or glycopeptide of claim 1, comprising the amino acid sequence set forth in any one of SEQ ID NOs: 1-32 or an amino acid sequence at least about 80-95% identical to the amino acid sequence set forth in any one of SEQ ID NOs: 1-32.

3. (canceled)

4. The isolated BclA3 glycoprotein or glycopeptide of claim 1, comprising one or more chain of three, five, or more N-acetyl hexosamine (HexNAc) moieties O-linked through a threonine residue.

5. (canceled)

6. (canceled)

7. The isolated BclA3 glycoprotein or glycopeptide of claim 4, wherein each HexNAc moiety has a molecular weight of about 203 Da, optionally wherein the BclA3 glycoprotein or glycopeptide further comprises a glycan capping moiety at the end of the HexNAc chain.

8. (canceled)

9. (canceled)

10. The isolated BclA3 glycoprotein or glycopeptide of claim 1, wherein the glycoprotein or glycopeptide has the nLC-MS/MS spectrum shown in any one of FIGS. 3a, 3b, 8a, and 8b.

11. An isolated C. difficile spore BclA3 glycan, comprising one or more chain of three or more N-acetyl hexosamine (HexNAc) moieties, optionally further comprising a glycan capping moiety at the end of the HexNAc chain.

12. (canceled)

13. (canceled)

14. (canceled)

15. The isolated BclA3 glycan of claim 11, wherein the BclA3 glycan is conjugated to a carrier molecule, the carrier molecule comprising a peptide, a protein, a membrane protein, a carbohydrate moiety, or a combination thereof, or a liposome containing any of the previous carrier molecules.

16. (canceled)

17. A composition comprising the isolated BclA3 glycoprotein or glycopeptide according to claim 1 and a pharmaceutically acceptable diluent, carrier, excipient, or adjuvant.

18. (canceled)

19. A conjugated BclA3 antigen comprising the isolated BclA3 glycoprotein or glycopeptide according to claim 1 conjugated to a carrier molecule.

20. (canceled)

21. (canceled)

22. (canceled)

23. A composition comprising the conjugated BclA3 antigen according to claim 19 and a pharmaceutically acceptable diluent, carrier, excipient, or adjuvant.

24. (canceled)

25. A composition comprising an antibody or fragment thereof that binds to a C. difficile spore glycoprotein or fragment thereof, wherein the glycoprotein or fragment thereof comprises BclA3 glycoprotein or a BclA3 glycopeptide according to claim 1; and a pharmaceutically acceptable diluent, carrier, or excipient.

26. The composition of claim 25, wherein the BclA3 glycoprotein or the glycopeptide comprises the amino acid sequence set forth in any one of SEQ ID NOs: 1-32, or an amino acid sequence at least about 80-95% identical to the amino acid sequence set forth in any one of SEQ ID NOs: 1-32.

27. (canceled)

28. The composition of claim 25, wherein the BclA3 glycoprotein or the glycopeptide comprises one or more chain of three, five, or more N-acetyl hexosamine (HexNAc) moieties O-linked through a threonine residue.

29.-34. (canceled)

35. A composition comprising an antibody or fragment thereof that binds to the C. difficile spore BclA3 glycan according to claim 11 and a pharmaceutically acceptable diluent, carrier, or excipient.

36. The composition of claim 35, wherein the BclA3 glycan comprises three or more N-acetyl hexosamine (HexNAc) moieties, optionally capped with a carbohydrate moiety having a molecular weight of about 203 Da, about 215 Da, about 220 Da, about 372 Da, 374 Da, 429 Da, 486 Da, 462 Da, 375 Da, 424 Da, or 552 Da.

37. The composition of claim 35, wherein the BclA3 glycan is conjugated to a carrier molecule, the carrier molecule comprising a peptide, a protein, a membrane protein, a carbohydrate moiety, or a combination thereof, or a liposome containing any of the previous carrier molecules.

38. (canceled)

39. An isolated antibody or fragment thereof specific for C. difficile spores, wherein the isolated antibody or fragment binds specifically to one or more of a BclA3 glycoprotein, a BclA3 glycopeptide, and a BclA3 glycan, wherein the BclA3 glycoprotein or the glycopeptide is as defined in claim 2.

40.-55. (canceled)

56. A method for preventing or treating C. difficile infection comprising administering to a subject the composition according to claim 17; such that C. difficile infection is prevented or treated in the subject.

57.-68. (canceled)

69. A method of detecting the presence of C. difficile in a subject comprising:
obtaining a stool sample from the subject; and
assaying the stool sample for the presence of a BclA3 glycoprotein, glycopeptide, and/or glycan thereof;
wherein the presence of the BclA3 glycoprotein, glycopeptide, and/or glycan thereof in the stool sample indicates the presence of C. difficile in the subject.

70.-72. (canceled)

73. A vaccine for prevention or treatment of C. difficile infection comprising the composition according to claim 17.


SEQUENCE LISTING (32 sequences)

SEQ ID NO: 1 - protein, 678 aa, Clostridium difficile
MSRNKYFGPFDDNDYNNGYDKYDDCNNGRDDYNSCDCHHCCPPSCVGPTGPMGPRGRTGPTGPTGPTGPGVGGTGPTGPTGPTGPTGNTGNTGATGLRGPTGATGGTGPTGATGAIGFGVTGPTGPTGPTGATGATGADGVTGPTGPTGATGADGITGPTGATGATGFGVTGPTGPTGATGVGVTGATGLIGPTGATGTPGATGPTGAIGATGIGITGPTGATGATGADGATGVTGPTGPTGATGADGVTGPTGATGATGIGITGPTGATGATGIGITGATGLIGPTGATGATGATGPTGVTGATGAAGLIGPTGATGVTGADGATGATGATGATGPTGADGLVGPTGATGATGADGLVGPTGPTGATGVGITGATGATGATGPTGADGLVGPTGATGATGADGVAGPTGATGATGNTGADGATGPTGATGPTGADGLVGPTGATGATGLAGATGATGPIGATGPTGADGATGATGATGPTGADGLVGPTGATGATGATGPTGPTGASAIIPFASGIPLSLTTIAGGLVGTPGFVGFGSSAPGLSIVGGVIDLTNAAGTLTNFAFSMPRDGTITSISAYFSTTAALSLVGSTITITATLYQSTAPNNSFTAVPGATVTLAPPLTGILSVGSISSGIVTGLNIAATAETRFLLVFTATASGLSLVNTVAGYASAGIAIN

SEQ ID NO: 2 - protein, 12 aa, Clostridium difficile
AGLIGPTGATGV

SEQ ID NO: 3 - protein, 15 aa, Clostridium difficile
TGPTGATGADGITGP

SEQ ID NO: 4 - protein, 9 aa, Clostridium difficile
VGPTGATGA

SEQ ID NO: 5 - protein, 14 aa, Clostridium difficile
GLIGPTGATGTPGA

SEQ ID NO: 6 - protein, 15 aa, Clostridium difficile
TGATGLIGPTGATGA

SEQ ID NO: 7 - protein, 14 aa, Clostridium difficile
TGIGITGPTGATGA

SEQ ID NO: 8 - protein, 11 aa, Clostridium difficile
TGIGITGPTGA

SEQ ID NO: 9 - protein, 14 aa, Clostridium difficile
GLIGPTGATGVTGA

SEQ ID NO: 10 - protein, 15 aa, Clostridium difficile
TGVTGATGAAGLIGP

SEQ ID NO: 11 - protein, 15 aa, Clostridium difficile
TGATGLIGPTGATGA

SEQ ID NO: 12 - protein, 18 aa, Clostridium difficile
IGPTGATGTPGATGPTGA

SEQ ID NO: 13 - protein, 15 aa, Clostridium difficile
TGPTGATGPTGADGL

SEQ ID NO: 14 - protein, 17 aa, Clostridium difficile
GVTGPTGPTGPTGATGA

SEQ ID NO: 15 - protein, 14 aa, Clostridium difficile
GVTGPTGPTGATGV

SEQ ID NO: 16 - protein, 15 aa, Clostridium difficile
VGPTGATGATGADGL

SEQ ID NO: 17 - protein, 12 aa, Clostridium difficile
VGPTGPTGATGV

SEQ ID NO: 18 - protein, 18 aa, Clostridium difficile
IGPTGATGTPGATGPTGA

SEQ ID NO: 19 - protein, 15 aa, Clostridium difficile
IGPTGATGVTGADGA

SEQ ID NO: 20 - protein, 12 aa, Clostridium difficile
VGPTGATGATGL

SEQ ID NO: 21 - protein, 15 aa, Clostridium difficile
VGPTGATGATGADGV

SEQ ID NO: 22 - protein, 12 aa, Clostridium difficile
VGPTGPTGATGV

SEQ ID NO: 23 - protein, 13 aa, Clostridium difficile
ATASGLSLVNTVA

SEQ ID NO: 24 - protein, 675 aa, Clostridium difficile
MSRNKYFGPFDDNDYNNGYDKYDDCNNGRDDYNSCDCHHCCPPSCVGPTGPMGPRGRTGPTGPTGPTGPGVGGTGPTGPTGPTGPTGNTGNTGATGLRGPTGATGGTGPTGATGAIGFGVTGPTGPTGATGATGADGVTGPTGPTGATGADGITGPTGATGATGFGVTGPTGPTGATGVGVTGATGLIGPTGATGTPGATGPTGAIGATGIGITGPTGATGATGADGATGVTGPTGPTGATGADGVTGPTGATGATGIGITGPTGATGATGIGITGATGLIGPTGATGATGATGPTGVTGATGAAGLIGPTGATGVTGADGATGATGATGATGPTGADGLVGPTGATGATGADGLVGPTGPTGATGVGITGATGATGATGPTGADGLVGPTGATGATGADGVAGPTGATGATGNTGADGATGPTGATGPTGADGLVGPTGATGATGLAGATGATGPIGATGPTGADGATGATGATGPTGADGLVGPTGATGATGATGPTGPTGASAIIPFASGIPLSLTTIAGGLVGTPGFVGFGSSAPGLSIVGGVIDLTNAAGTLTNFAFSMPRDGTITSISAYFSTTAALSLVGSTITITATLYQSTAPNNSFTAVPGATVTLAPPLTGILSVGSISSGIVTGLNIAATAETRFLLVFTATASGLSLVNTVAGYASAGIAIN

SEQ ID NO: 25 - protein, 12 aa, Clostridium difficile
TGPTGVTGATGA

SEQ ID NO: 26 - protein, 14 aa, Clostridium difficile
GVTGPTGPTGATGA

SEQ ID NO: 27 - protein, 14 aa, Clostridium difficile
GVTGPTGPTGATGV

SEQ ID NO: 28 - protein, 9 aa, Clostridium difficile
TGPTGADGL

SEQ ID NO: 29 - protein, 14 aa, Clostridium difficile
GLVGPTGPTGATGV

SEQ ID NO: 30 - protein, 18 aa, Clostridium difficile
AGPTGATGATGNTGADGA

SEQ ID NO: 31 - protein, 28 aa, Clostridium difficile
TGPTGATGPTGADGLVGPTGATGATGLA

SEQ ID NO: 32 - protein, 15 aa, Clostridium difficile
IGPTGATGVTGADGA